(19) United States
(12) Patent Application Publication
(10) Pub. No.: US 2025/0222091 A1
(43) Pub. Date: Jul. 10, 2025

(54) POLYVALENT VACCINES FOR STAPHYLOCOCCAL & STREPTOCOCCAL INFECTION

(71) Applicant: University of Tennessee Research Foundation, Knoxville, TN (US)

(72) Inventor: Oudessa Kerro Dego, Knoxville, TN (US)

(73) Assignee: University of Tennessee Research Foundation, Knoxville, TN (US)

(21) Appl. No.: 19/089,742

(22) Filed: Mar. 25, 2025

Related U.S. Application Data
(63) Continuation of application No. PCT/US2023/034010, filed on Sep. 28, 2023.
(60) Provisional application No. 63/377,454, filed on Sep. 28, 2022.

(51) Int. Cl. A61K 39/09; A61K 39/00; A61K 39/085; A61P 37/04

(52) U.S. Cl. CPC A61K 39/092; A61K 39/085; A61P 37/04; A61K 2039/552; A61K 2039/70

(57) ABSTRACT

Disclosed are methods of treating and/or inhibiting an intramammary infection in a mammalian subject. The methods included administering a therapeutically effective amount of surface-associated polypeptides from various bacteria. Also disclosed are methods for blocking or reducing infection in a mammal. Further disclosed are methods for reducing the rate of intra-mammary infection and/or mastitis in a dairy mammal. Methods of increasing milk production are also provided.

### REFERENCE TO RELATED APPLICATIONS

This application is a continuation application of 35 U.S.C. § 120 of International Application No. PCT/US2023/034010, filed 28 Sep. 2023, which claims priority under to U.S. Provisional Application No. 63/377,454, filed Sep. 28, 2022 each of which is incorporated by reference in its entirety.

### TECHNICAL FIELD

Disclosed are compositions and methods related to the prevention of mastitis. The polyvalent vaccines provided herein reduce Staphylococcal or Streptococcal infections that can result in clinical and subclinical mastitis. Methods of increasing milk yield are also provided. More specifically, the present disclosure provides surface-associated polypeptides that can inhibit bacterial infection.

### BACKGROUND

Mastitis is an inflammation of mammary glands usually caused by bacteria, fungi, yeast or algae. Mastitis is estimated to cost the United States (U.S.) dairy industry more than $2 billion annually (NMC, 1996, Rollin, Dhuyvetter and Overton, 2015, Liang et al., 2017). On average, clinical mastitis costs $444 per case during the first 30 days in milk (DIM) (Rollin, Dhuyvetter and Overton, 2015). Staphylococcus aureus (S. aureus), non-aureus staphylococci (NAS) and Streptococcus uberis (S. uberis) are major Gram-positive bacteria that cause mastitis in dairy cows. Staphylococci that cause mastitis in dairy cows are 1) Staphylococcus aureus and 2) non-aureus staphylococci (NAS). Staphylococcus aureus is a major contagious mammary pathogen on U.S. dairy farms (APHIS, 2008). Non-aureus staphylococci are a group of more than 50 species (Becker, Heilmann and Peters, 2014, Condas et al., 2017a) comprised of some species increasingly reported as a cause of subclinical mastitis in dairy cows (Gillespie et al., 2009, Condas et al., 2017a, Jenkins et al., 2019).

Staphylococcus chromogenes is a predominant NAS (Gillespie et al., 2009, Condas et al., 2017b, Jenkins et al., 2019) consistently isolated from subclinical mastitis cases (Taponen and Pyorala, 2009, Vanderhaeghen et al., 2015), cow's udder, and teat skin (Taponen, Bjorkroth and Pyorala, 2008, Thorberg et al., 2009). A study on 5, 710 quarter milk samples from 1847 cows with high SCC from 30 dairy herds in the Southeast Region (TN, KY, MS & VA) of the United States found NAS prevalence of 38.9%, S. aureus 15.6%, S. dysagalactiae 7.6%, E. coli 5.3% and S. uberis 4.1% (Pighetti et al., 2018). Of the NAS isolates, S. chromogenes represented the dominant NAS representing 67% of the isolates (Pighetti et al., 2018). Similarly, a study on conventional and organic Canadian dairy farms found NAS in 20% of the clinical samples (Levison et al., 2016) and in 43% of quarters with SCC <200,000 cells/ml (Reyher et al., 2011, Dufour et al., 2012). Mammary glands pre-colonized by certain strains of NAS will not be infected by major mastitis bacteria (De Vliegher et al., 2004, Stevens et al., 2018). Some strains of S. chromogenes also inhibited in vitro growth of major mastitis pathogens, such as S. aureus, S. uberis, and S. dysgalactiae, via the production of bacteriocin (De Vliegher et al., 2004, Pyorala and Taponen, 2009). Even though pre-colonization of udder by some NAS seems protective against colonization by major mastitis pathogens, the NAS themselves are increasingly reported as etiology of mastitis and other diseases of animals and humans (De La Fuente, Suarez and Schleifer, 1985, Roberson et al., 1996, Devriese et al., 2005, Götz, Bannerman and Schleifer, 2006, Bannoehr et al., 2007, Sasaki et al., 2007, Markey, 2013, De Buck et al., 2021). So, NAS should be considered opportunistic pathogens of animals and humans. S. chromogenes is consistently isolated from the cow's udder and teat skin (Taponen et al, 2008) and causes long-lasting, persistent subclinical infections (Taponen and Pyorala, 2009). Farm data also showed that NAS are more prevalent in farms with low bulk tank milk SCC (Schukken et al., 2009).

Current mastitis control programs are not fully effective and antibiotics are not sustainable because of limited success (Barkema et al 2006; McDougall et al 2009). Management based mastitis control measures have been developed and implemented with limited success in reducing contagious bacteria such as S. aureus and S. agalactiae (Bekuma and Galmessa, 2018, Bhakat et al., 2020, Rowe, Tranter and Laven, 2021), but poor success due to the application disparities across mastitis management (Stevens, Piepers and De Vliegher, 2019). Dependence on antibiotics to control S. aureus mastitis is not sustainable due to limited success (Barkema, Schukken and Zadoks, 2006, McDougall et al., 2009) and the emergence of bacteria resistant to the commonly used antimicrobials (Sawant, Gillespie and Oliver, 2009, Abdi et al., 2018)

Currently, there is one commercial bacterin vaccine claimed to have some effects against S. aureus mastitis in dairy cows in the United States. Studies evaluating the efficacy of this commercial vaccine found no significant difference between vaccinated and unvaccinated control cows (Luby and Middleton, 2005, Middleton et al., 2006, Luby et al., 2007). Another polyvalent commercial bacterin vaccine containing inactivated high biofilm-forming S. aureus strain SP 140 and E. coli J5 strain is available in Europe and some other countries for the control of mastitis caused by S. aureus, NAS, E. coli, and other coliforms in dairy cows. Some efficacy studies on this vaccine concluded that vaccination with the polyvalent bacterin reduced mastitis incidence, severity, and duration (Schukken et al., 2014, Bradley et al., 2015, Piepers et al., 2017) whereas others concluded that vaccination with the polyvalent bacterin did not induce a significant reduction in staphylococcal intramammary infection (IMI) between vaccinated and unvaccinated groups (Prenafeta et al., 2010, Landin et al., 2015, Freick et al., 2016, Tashakkori et al., 2020). Freick et al. found a significantly lower SCC in the Bestvac vaccinated group compared to the unvaccinated group (Freick et al., 2016). Based on published vaccine efficacy studies in the United States, currently available vaccines cannot be recommended as part of the routine measures for controlling S. aureus mastitis.

S. uberis is frequently isolated from the dairy farm environment and remarkably adaptable to environmental changes (Zadoks et al., 2005). S. uberis causes intramammary infection (IMI) of lactating, dry, heifers, and multiparous cows throughout the year. Streptococcus uberis IMI can become clinical or subclinical mastitis, which in some cases may lead to persistent IMI without an increase in the somatic cell count (SCC) (Oliver et al., 1998, Zadoks et al., 2003). Some studies showed that S. uberis strains from IMI are host-adapted and have characteristics of contagious nature, whereas other strains are not host-adapted and cause transient IMI of environmental nature (Zadoks et al., 2003, Tassi et al., 2013).

Despite several years of vaccine trials, there is no effective commercial vaccine against S. uberis mastitis in dairy cows that can prevent clinical disease and associated production losses. The only commercial vaccine UBAC® S. uberis vaccine (UBAC®) (Hipra, Amir, Spain) that exists in Europe, Canada, and few other countries, achieved some level of efficacy (partial efficacy) but did not prevent clinical disease and production losses. There is no vaccine against S. uberis mastitis in the United States. The UBAC® S. uberis vaccine from Hipra is also not well characterized under controlled experimental studies and field-based studies to confirm label claims from the producer.

Intramammary vaccinations of dairy cows with bacterin induced protection from experimental infection with the same strain (Finch et al., 1994) but were less effective when challenged with heterologous strain (Finch et al., 1997). Subcutaneous injection of live S. uberis with subsequent booster injection with S. uberis cell surface proteins through intramammary route induced protection against homologous strain but the efficacy was limited against heterologous strain (Finch et al., 1997). Vaccination with multifunction protein (adhesin and glycolytic) S. uberis glyceraldehyde-3-phosphate dehydrogenase C (GapC) reduced inflammation post-challenge (Fontaine et al., 2002). Some of the technical aspects of the challenge protocols (Fontaine et al., 2002) were questionable (Leigh, 2002). Although GapC is a highly immunogenic protein, its protective effect as vaccine antigen is yet to be determined. The pauA gene is a plasminogen activator (Lincoln and Leigh, 1997) that was expected to promote bacterial invasion into a host tissue (Leigh and Lincoln, 1997). A vaccination trial with PauA protein induces increased antibodies that achieved partial protection (Leigh et al., 1999). The growth of pauA deletion mutant clone of S. uberis in milk or its ability to infect the udder of lactating dairy cows did not change. It was concluded that PauA protein does not have a role in the S. uberis IMI (Ward et al., 2003).

S. uberis has multiple proteins on its cell surface including S. uberis adhesion molecule (SUAM) and extracellular proteins that bind to host matrix, which allows bacterial adhesion and invasion of udder tissue, resulting in the establishment of IMI (Almeida et al., 1996, Almeida et al., 1999, Almeida and Oliver, 2001, Almeida et al., 2006, Almeida and Oliver, 2006, Patel et al., 2009, Almeida et al., 2010, Almeida et al., 2015a). Recombinant SUAM (rSUAM) based vaccine efficacy trials in dairy cows induced good immunological responses in vaccinates compared with unvaccinated controls (Prado et al., 2011). Under in vitro study, the hyperimmune serum from rSUAM vaccinated cows reduced S. uberis attachment and internalization into epithelial cells (Prado et al., 2011). Intramammary infusion of S. uberis co-incubated with hyperimmune serum from rSUSAM vaccinates reduced the severity of the disease (Almeida et al., 2015b). The sua gene mutant clone is less virulent to epithelial cells (Chen et al., 2011) and cows (Almeida et al., 2015a). Series of controlled experimental vaccination of cows with rSUSAM and subsequent challenge with heterologous strains showed that rSUAM is immunogenic (Prado et al., 2011). Similarly, intramammary administration of S. uberis preincubated with hyperimmune serum from rSUAM vaccinated cows reduced clinical mastitis and bacterial shedding through milk, post-challenge.

Vaccination trials with a single protein (subunit vaccine) such as S. uberis glyceraldehyde-3-phosphate dehydrogenase C (GapC) (Fontaine et al., 2002), plasminogen activator A (PauA) (Leigh et al., 1999), rSUAM (Prado et al., 2011) induced increased immunity but the protective efficacy of induced immunity against S. uberis mastitis is limited.

There is no efficacious vaccine against S. uberis mastitis in dairy cows. Consequently, the control of S. uberis mastitis is based on good management practices such as maintaining clean and dry housing areas, culling chronic cases, dry cow therapy, and treating clinical cases. There is critical need to develop effective vaccine that prevents clinical Strep. uberis mastitis and associated production losses.

Virulence factors of S. uberis are not well understood. Encapsulation and biofilm formation (Matthews and Oliver, 1993, Matthews et al., 1994, Ward et al., 2001, Crowley et al., 2011, Varhimo et al., 2011, Kaczorek et al., 2017), adherence and invasion of mammary epithelial cells (Almeida et al., 1996, Almeida et al., 2006), virulence-associated immunogenic surface proteins (Kerro Dego et al., 2018), and other virulence factors (Ward et al., 2009, Egan et al., 2012, Gunther et al., 2016) appear to be associated with the disease process. An exhaustive search for a single virulence factor as a vaccine candidate seems to be of limited success.

Some efficacy studies on a commercial bacterin vaccine for S. aureus mastitis in the United States showed that vaccination of dairy cows with this commercial vaccine containing different strains of S. aureus reduced incidence, SCC, and clinical mastitis (Williams, Mayerhofer and Brown, 1966, Williams et al., 1975, Nickerson et al., 1999). In contrast, efficacy studies on this bacterin vaccine by others (Luby and Middleton, 2005, Middleton et al., 2006, Smith, Lyman and Anderson, 2006, Luby et al., 2007, Middleton, Luby and Adams, 2009) concluded some reduction in severity and incidence but little or no protection effects. Similarly, some efficacy studies on another commercial bacterin vaccine for S. aureus, CNS, and E. coli available in Europe and some other countries showed that this vaccine has no effect on the incidence of cases but reduced the severity and duration of staphylococcal mastitis (Schukken et al., 2014, Bradley et al., 2015, Piepers et al., 2017). In contrast, others concluded that this vaccine has no protective effects against S. aureus (Prenafeta et al., 2010, Landin et al., 2015, Freick et al., 2016). Overall, most studies on these two commercial bacterin vaccines agree that vaccination with these vaccines increases antibody titers in milk and serum, but neither vaccine significantly exhibited dependable protection against staphylococcal IMI (Middleton et al., 2006, Nickerson et al., 2008, Schukken et al., 2014). Other S. aureus protein A (Pankey et al., 1985) and S. aureus fibronectin-binding protein (FnBP)-clumping factor A (CIfA) (Shkreta et al., 2004) vaccines conferred increased spontaneous cure after experimental challenges, but none of them were tested under field conditions.

One of the major constraints is identifying protective immune responses that can be induced in the mammary gland by vaccination. All vaccination efforts are trial and error without knowing which immune type or types can achieve protection against staphylococcal mastitis in the mammary glands.

### SUMMARY OF THE INVENTION

Compositions and methods pertaining to Staphylococcus aureus associated conditions, Staphylococcus chromogenes associated conditions, and Streptococcus uberis associated conditions are provided herein. In an embodiment, a pharmaceutical composition comprising isolated surface-associated polypeptides, wherein said surface associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP) is provided. In an aspect, the pharmaceutical composition comprises at least two isolated surface associated polypeptides selected from the group consisting of Staphylococcus aureus surface associated polypeptides (SASP), Staphylococcus chromogenes surface associated polypeptides (SCSP) and Streptococcus uberis surface associated polypeptides (SUSP). In an aspect, the pharmaceutical composition comprises isolated SASP, SCSP and SUSP. In various aspects, the composition further comprises an adjuvant capable of stimulating the immune response in a dairy producing mammal. In an aspect, the pharmaceutical composition is for use in a dairy producing mammal selected from the group consisting of dairy cows, sheep, goats, camels, buffaloes, yaks, horses, reindeer and donkeys. In other aspects, the pharmaceutical composition is for use in a human to control Staphylococcus aureus infections and or Streptococcal infections, optionally Streptococcus pneumoniae, Streptococcus pyogenes or Streptococcus agalactiae.

In various aspects of the pharmaceutical compositions, the isolated surface associated polypeptides are present in a therapeutically effective amount. In certain aspects, the therapeutically effective amount is sufficient to inhibit whole cow mastitis in a subject exposed to bacteria. In some aspects of the pharmaceutical compositions, the pharmaceutical composition comprises at least about 0.05 mg SASP. In some aspects of the pharmaceutical composition, the pharmaceutical composition comprises at least about 0.05 mg SCSP. In some aspects of the pharmaceutical compositions, at least two isolated surface-associated proteins are present at a pre-determined ratio.

In certain aspects, the pharmaceutical composition confers long-lasting resistance to an intramammary infection (IMI). In some aspects, the pharmaceutical composition inhibits bacterial infection in a subject to which the composition has been administered. In certain aspects, the bacteria is selected from the group consisting of Staphylococcus spp and Streptococcus spp.

In various aspects of the pharmaceutical compositions, the surface associated polypeptides are present in an effective amount to reduce the somatic cell count (SCC) in milk from a subject treated with the pharmaceutical composition.

In aspects of the pharmaceutical composition, a subject to whom the pharmaceutical composition has been administered exhibits reduced risk of a disorder selected from the group consisting of intramammary infection, clinical mastitis, subclinical mastitis and persistent intramammary infection.

In various aspects, the pharmaceutical composition is suitable for injection, optionally subcutaneous injection or intramammary gland injection.

In an embodiment, the application provides a polyvalent vaccine against intramammary infection comprising at least two surface-associated polypeptides selected from the group consisting of Staphylococcus aureus surface associated polypeptides (SASP), Staphylococcus chromogenes surface associated polypeptides (SCSP) and Streptococcus uberis surface associated polypeptides (SUSP). In an aspect of the vaccine, the isolated surface associated polypeptides are present at a predetermined ratio. In certain aspects, the polyvalent vaccine comprises Staphylococcus aureus surface associated polypeptides (SASP), Staphylococcus chromogenes surface associated polypeptides (SCSP) and Streptococcus uberis surface associated polypeptides (SUSP). In various aspects, the ratio of surface-associated polypeptides is approximately 1:1:1. In other aspects, the ratio of surface-associated polypeptides is not 1:1:1. In some aspects, the polyvalent vaccine comprises between about 50 μg and about 5000 μg surface-associated polypeptides. In other aspects, the polyvalent vaccine comprises between about 100 μg and about 3000 μg surface-associated polypeptides. In certain aspects, the polyvalent vaccine comprises between about 150 μg and about 2000 μg surface-associated polypeptides.

In various aspects of the polyvalent vaccine, the vaccine further comprises an adjuvant capable of stimulating the immune response in a dairy producing mammal. In certain aspects, the adjuvant is selected from the group comprising Emulsigen-D®, dimethyl-dioctadecyl ammonium bromide (DDA), and Montanide ISA 61VG®. In some aspects, the dairy producing mammal is selected from the group consisting of dairy cows, sheep, goats, camels, buffaloes, yaks, horses, reindeer and donkeys.

In an embodiment, methods of inhibiting bacterial infection in a subject are provided. The methods of inhibiting bacterial infection comprise administering a pharmaceutical composition comprising isolated surface-associated polypeptides to a subject, wherein the surface-associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP). Various aspects of the methods comprise administering a pharmaceutical composition comprising at least two isolated surface-associated polypeptides selected from the group consisting of Staphylococcus aureus surface associated polypeptides (SASP), Staphylococcus chromogenes surface associated polypeptides (SCSP) and Streptococcus uberis surface associated polypeptides (SUSP). Certain aspects of the methods comprise administering a pharmaceutical composition comprising isolated Staphylococcus aureus surface associated polypeptides (SASP), Staphylococcus chromogenes surface associated polypeptides (SCSP) and Streptococcus uberis surface associated polypeptides (SUSP). In various aspects of the methods, at least two isolated surface-associated polypeptides are present at a predetermined ratio.

In some aspects of the methods, administering comprises injecting the pharmaceutical composition, optionally subcutaneously injecting the pharmaceutical composition. Certain aspects of the methods comprise administering at least two doses of the pharmaceutical composition according to a dosing regimen, optionally comprising administering 2 to 5 doses of the pharmaceutical composition.

In various aspects of the methods, the subject is a dairy producing mammal. In certain aspects, the dairy producing mammal is selected from the group consisting of dairy cows, sheep, goats, camels, buffaloes, yaks, horses, reindeer and donkeys.

In some aspects of the methods, the bacterial infection is an intramammary infection. In certain aspects, the intramammary infection is persistent intramammary infection (IMI), clinical mastitis or subclinical mastitis.

In an embodiment, methods of reducing mastitis in a subject are provided. Methods of reducing mastitis comprise administering a pharmaceutical composition comprising isolated surface-associated polypeptides to the subject, wherein the surface-associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP). Various aspects of the methods comprise administering multiple doses of the pharmaceutical composition to the subject. Other aspects of the methods comprise administering a single dose of the pharmaceutical composition to the subject. In certain aspects of the methods, the subject exhibits a decreased risk of clinical mastitis after administration of the pharmaceutical composition. In some aspects of the methods, the subject exhibits a decreased risk of subclinical mastitis after administration of the pharmaceutical composition. In aspects of the methods, the subject exhibits decreased risk of subclinical mastitis at the quarter level after administration of the pharmaceutical composition. In aspects of the methods, the subject exhibits decreased risk of subclinical mastitis at the cow level.

Methods of increasing antibody titers in milk are provided. The methods comprise administering a pharmaceutical composition comprising isolated surface-associated polypeptides to a milk producing subject, wherein the surface-associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP).

In an embodiment, methods of reducing somatic cell count (SCC) in milk are provided. The methods comprise administering a pharmaceutical composition comprising isolated surface-associated polypeptides to a milk producing subject, wherein the surface-associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP).

In an embodiment, methods of reducing the incidence of subclinical mastitis in a herd of dairy producing mammals are provided. The methods comprise the steps of (a) providing a herd of dairy producing mammals and (b) administering a pharmaceutical composition comprising isolated surface associated polypeptides to members of the herd, wherein the surface associated polypeptides are selected from the group consisting of administering a pharmaceutical composition comprising isolated surface-associated polypeptides to a milk producing subject, wherein the surface-associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP).

In an embodiment, methods of increasing milk yield in a dairy mammal are provided. The methods comprising the steps of (a) providing a dairy mammal and (b) administering a pharmaceutical composition comprising isolated surface associated polypeptides to the dairy mammal, wherein the surface associated polypeptides are selected from the group consisting of administering a pharmaceutical composition comprising isolated surface-associated polypeptides to a milk producing subject, wherein the surface-associated polypeptides are selected from the group consisting of Staphylococcus aureus surface associated polypeptides (SASP), Staphylococcus chromogenes surface associated polypeptides (SCSP) and Streptococcus uberis surface associated polypeptides (SUSP).

### BRIEF DESCRIPTION OF FIGURES

FIG. 1 summarizes results obtained from SASP and SCSP vaccines against experimentally induced and naturally occurring staphylococcal mastitis in dairy cows. FIG. 1A provides a chart summarizing the results of experimental vaccination and challenge studies during the dry period. FIG. 1B provides a chart summarizing the results of experimental vaccination and challenge studies during early lactation. For FIGS. 1A and 1B, the numbers indicate the number of cows that exhibited the indicated status (SCM-subclinical mastitis; CM-clinical mastitis; NM-no mastitis. During the dry period (FIG. 1A), all cows in the control group exhibited either subclinical mastitis or clinical mastitis. Cows treated with the SASP vaccine were split evenly between subclinical mastitis and clinical mastitis, with one cow exhibiting no mastitis. All the cows treated with SCSP vaccine exhibited no mastitis during the dry period. During the early lactation period (FIG. 1B), the cows in the control group exhibited either subclinical mastitis (5), clinical mastitis (2) or no mastitis (3). The cows treated with SCSP vaccine exhibited either subclinical mastitis (2) or no mastitis (9) during the early lactation period.

FIG. 1C provides a chart summarizing the results of experimental vaccination and efficacy against naturally occurring staphylococcal subclinical mastitis over 300 days of lactation at the cow level. The SCSP vaccine treated cows exhibited a significantly lower incidence of naturally occurring staphylococcal subclinical mastitis than control cows. The SASP vaccine treated cows exhibited a substantially lower incidence of naturally occurring staphylococcal subclinical mastitis than control cows. FIG. 1D provides a chart summarizing milk yield from vaccinated and unvaccinated cows over 300 days of lactation. Days in milk is indicated on the x axis, milk production (kg) is on the y axis. Data from the control group is indicated with circles. Data from cows treated with SASP vaccines are indicated with squares. Data from cows treated with SCSP vaccines are indicated with triangles. FIG. 1E provides a chart summarizing milk yield during peak production time, milk yield from SASP and SCSP vaccinated cows was greater than milk yield from unvaccinated cows. Days in milk (DIM) is indicated on the x-axis; milk production (lb) is on the y axis.

FIG. 2 provides a chart indicating dairy cow susceptibility to intramammary infection (IMI) during the early dry period and transition period (adopted from Bradley and Green 2004 (Bradley and Green, 2004)). The early dry and transition periods are indicated by arrows. The line traces the rate of new infection. Dairy cows are highly susceptible to intramammary infection during the early dry and transition periods (peaks. In some instances, vaccination dosing regimens and challenge infection timelines were developed based on this time line. Potential vaccination dosing days are indicated with V1, V2 or V3. Challenge day is indicated also.

FIGS. 3A-3L present information relating to various Staphylococcus and Streptococcus surface protein preparations. FIGS. 3A, 3B and 3C provide images of polyacrylamide gels with samples of either Staphylococcus aureus surface proteins (SASP, FIG. 3A), Staphylococcus chromogenes surface proteins (SCSP, FIG. 3B) or Streptococcus uberis surface proteins (SUSP, FIG. 3C). FIG. 3D is an image of a Western blot of SUSP probed with immune serum from SUSP vaccinated cow. FIG. 3E is an image of a Western blot of SASP probed with immune serum from SASP vaccinated cow. FIG. 3F presents a graph summarizing the serum immune response (anti-SUSP IgG titer as a function of optical density, y-axis) after vaccination with different doses of SUSP. The timeline is on the x-axis. Circles represent control treatment, squares represent treatment with 100 μg SUSP, triangles represent treatment with 1 mg SUSP, diamonds represent treatment with 4 mg SUSP. Data are presented as a mean of each group, and error bars are 95% confidence intervals of the mean values. V1-V3: first to third vaccinations; DO: at drying off; D14, D28 & D42: 14, 28 & 42 days after drying off; C: at calving; C7, C14 & C28: 7, 14 & 28 days after calving; Ch0: Immediately before the challenge; Ch7, Ch14, Ch21 & Ch28: 7, 14, 21 & 28 days after challenge.

FIG. 3G presents a graph summarizing the somatic cell count (SCC) in milk from S. uberis challenged and unchallenged quarters of dairy cows vaccinated with the indicated SUSP dose. Day post challenge is presented on the x-axis; log 10 SCC is shown on the y-axis. Data are presented as log 10 of the actual counts, and error bars represent 95% confidence intervals for the mean values of all the challenged quarters (Yes) and unchallenged quarters (No) per group. Data from control cows are indicated with circles; data from cows treated with 100 μg SUSP are represented with squares; data from cows treated with 1 mg SUSP are represented with triangles; data from cows treated with 4 mg SUSP are represented with diamonds.

FIGS. 3H, 3I, 3J and 3K provide charts summarizing quarter-level mastitis status of different SUSP doses vaccinated groups. FIG. 3H presents results from cows treated with high dose, 4 mg SUSP. FIG. 3I presents results from cows treated with medium dose, 1 mg SUSP. FIG. 3J presents results from cows treated with low dose, 100 μg SUSP. FIG. 3K present results from cows treated with the control. Negative quarters (Neg, empty bars), quarters with subclinical (SC, bars) mastitis, and quarters with clinical (CL, bars) mastitis. Data were presented as total numbers of negative, subclinical, or clinical quarters per group at each sampling time point (challenge day (Chn), x-axis).

FIG. 3L provides a summary of S. uberis strain UT888 counts in CFU/mL of milk from different SUSP treatment groups. The S. uberis strain UT888 counts in CFU/mL in log 10 is shown on the y-axis. The SUSP treatment group is indicated on the x-axis. Data are presented as the mean of log 10 CFU/mL per group at each time point, and error bars represent the 95% confidence intervals. Different letters show statistically (P<0.05) different counts.

FIGS. 4A and 4B present charts summarizing the rectal temperature and vaccine injection site reaction observed in cows after subcutaneous injection of either a control (circles), SCSP (triangles) or SASP (SASP). Days in milk are shown on the x-axes in both FIGS. 4A and 4B. Rectal temperature is shown on the y-axis in FIG. 4A; reaction site volume (cm³) is shown on the y-axis in FIG. 4B. Data are presented as a mean of each group, and error bars are 95% confidence intervals of the mean values. SASP: Staphylococcus aureus surface associated proteins; SCSP: S. chromogenes surface associated proteins; −59:59 days before expected calving date; −35:35 days before expected calving date.

FIGS. 5A-5D present graphs summarizing the mean serum anti-SASP titers after vaccination with either a control (circles), SCSP (triangles) or SASP (SASP). Days in milk are shown on the x-axes of FIGS. 5A, 5B, 5C and 5D. −60: 60 days before expected calving date; −40:40 days before expected calving; −20:20 days before expected calving; 0: calving day, V1-V3: 1^{st} to 3^{rd} vaccinations, V4 and V5: 4^{th} and 5^{th} booster vaccinations. FIG. 5A summarizes mean anti-SASP IgA titers (y-axis). FIG. 5B summarizes mean anti-SASP IgG titers (y-axis). FIG. 5C summarizes mean anti-SASP IgG1 titers (y-axis). FIG. 5D summarizes mean anti-SASP IgG2 titers (y-axis). Data are presented as the mean of base 10 log antibody titers, and error bars are 95% confidence intervals of the mean values of each group. Single asterisks indicate significantly higher serum anti-SASP antibody titers in SASP vaccinated cows compared to that control cows, whereas double asterisks indicate significantly higher cross-reacting serum anti-SCSP antibody titers in SASP vaccinated cows compared to control cows.

FIGS. 6A-6D present graphs summarizing the mean serum anti-SCSP titers after vaccination with either a control (circles), SCSP (triangles) or SASP (SASP). Days in milk are shown on the x-axes of FIGS. 6A, 6B, 6C and 6D. −60: 60 days before expected calving date; −40:40 days before expected calving; −20:20 days before expected calving; 0: calving day, V1-V3: 1^{st} to 3^{rd} vaccinations, V4 and V5: 4^{th} and 5^{th} booster vaccinations. FIG. 6A summarizes mean anti-SCSP IgA titers (y-axis). FIG. 6B summarizes mean anti-SCSP IgG titers (y-axis). FIG. 6C summarizes mean anti-SCSP IgG1 titers (y-axis). FIG. 6D summarizes mean anti-SCSP IgG2 titers (y-axis). Data are presented as the mean of base 10 log antibody titers, and error bars are 95% confidence intervals of the mean values of each group. Black asterisks show significantly higher serum anti-SCSP antibody titers in SCSP vaccinated cows compared to that control cows whereas red asterisks show significantly higher cross-reacting serum anti-SASP antibody titers in SCSP vaccinated cows compared to control cows.

FIGS. 7A-7D present graphs summarizing the mean milk anti-SASP titers after vaccination with either a control (circles), SCSP (triangles) or SASP (SASP). Days in milk are shown on the x-axes of FIGS. 7A, 7B, 7C and 7D. −60: 60 days before expected calving date; 0: calving day, V1-V3: 1^{st} to 3^{rd} vaccinations, V4 and V5: 4^{th} and 5^{th} booster vaccinations. FIG. 7A summarizes mean anti-SASP IgA titers (y-axis). FIG. 7B summarizes mean anti-SASP IgG titers (y-axis). FIG. 7C summarizes mean anti-SASP IgG1 titers (y-axis). FIG. 7D summarizes mean anti-SASP IgG2 titers (y-axis). Data are presented as the mean of base 10 log antibody titers, and error bars are 95% confidence intervals of the mean values of each group. Red asterisks show significantly higher milk anti-SASP antibody titers in SASP vaccinated cows compared to control cows, whereas black asterisks show significantly higher cross-reacting milk anti-SCSP antibody titers in SASP vaccinated cows compared to that control cows. Samples at 5 time points were analyzed.

FIGS. 8A-8D present graphs summarizing the mean milk anti-SCSP titers after vaccination with either a control (circles), SCSP (triangles) or SASP (SASP). Days in milk are shown on the x-axes of FIGS. 8A, 8B, 8C and 8D. −60: 60 days before expected calving date; 0: calving day, V1-V3: 1^{st} to 3^{rd} vaccinations, V4 and V5: 4^{th} and 5^{th} booster vaccinations. FIG. 8A summarizes mean anti-SCSP IgA titers (y-axis). FIG. 8B summarizes mean anti-SCSP IgG titers (y-axis). FIG. 8C summarizes mean anti-SCSP IgG1 titers (y-axis). FIG. 8D summarizes mean anti-SCSP IgG2 titers (y-axis). Data are presented as the mean of base 10 log antibody titers, and error bars are 95% confidence intervals of the mean values of each group. The asterisk shows significantly higher cross-reacting milk anti-SASP antibody titers in SCSP vaccinated cows compared to that of control cows.

FIG. 9 presents a graph summarizing the effect of vaccination on mean counts of non-aureus staphylococci by vaccine group over 300 days of lactation. Data are presented as the mean of base 10 log CFU of non-aureus staphylococci (NAS)/mL per treatment group, and error bars represent the 95% confidence intervals of mean values. SASP: Staphylococcus aureus surface associated proteins; SCSP: S. chromogenes surface associated proteins. Treatment group is indicated on the x-axis.

FIGS. 10A and 10B present graphs summarizing the effect of vaccination on the cumulative incidence of cow-level (FIG. 10A) and quarter-level (FIG. 10B) subclinical mastitis over 300 days of lactation. Groups indicated with different letters are significantly different. Results from the control group (control), SASP treated (SASP) and SCSP treated (SCSP) cows are shown. Incidence (proportion) is shown on the y-axes.

FIG. 11 provides a chart summarizing the incidence density of subclinical mastitis at the quarter level in cows treated with either a control, SASP or SCSP. Both SASP and SCSP treated cows exhibited a significant decrease in incidence density of subclinical mastitis at the quarter level as compared to cows treated with a control.

FIG. 12 provides a chart summarizing the vaccine efficacy of SASP and SCSP. SCSP treated cows were 10× less likely to develop SCM than control treated cows.

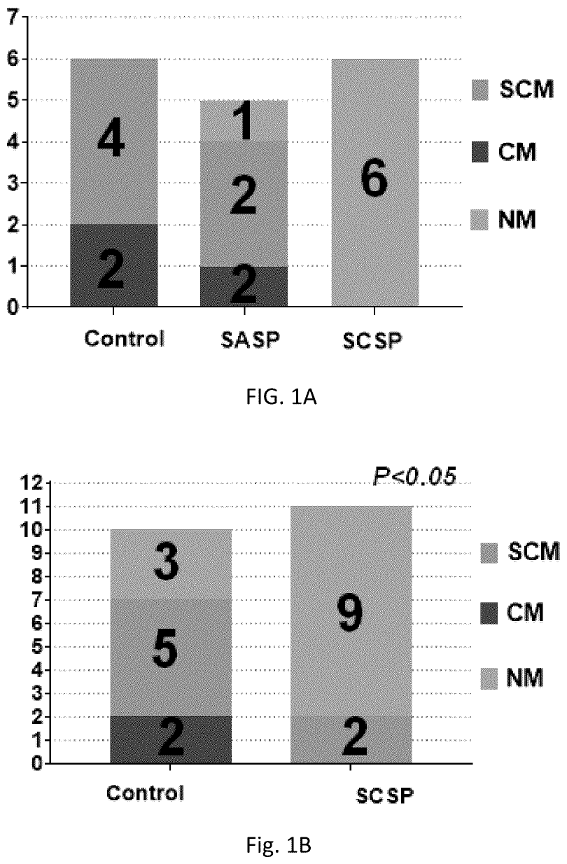

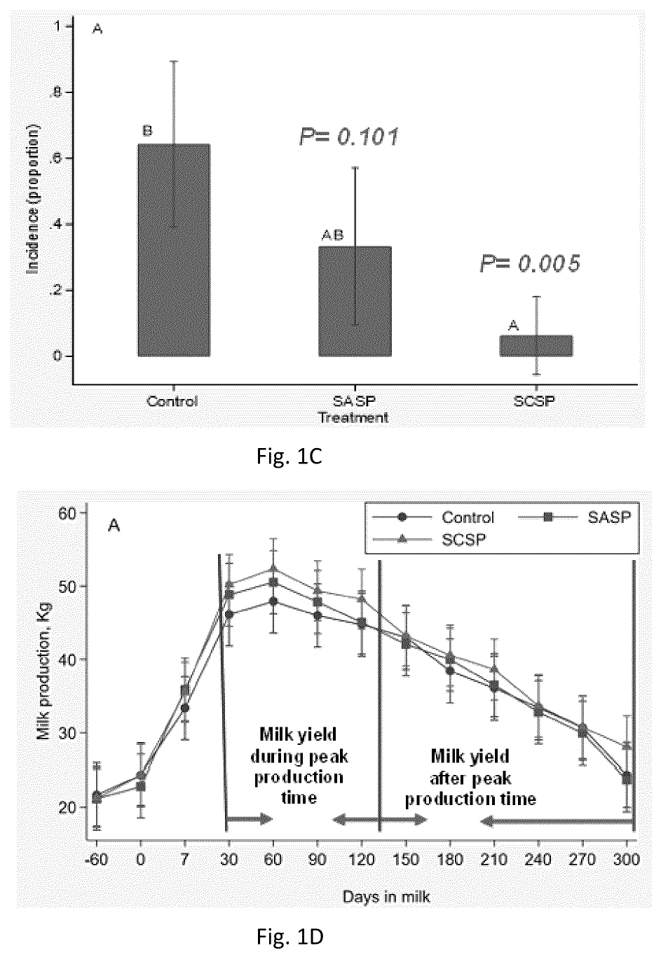

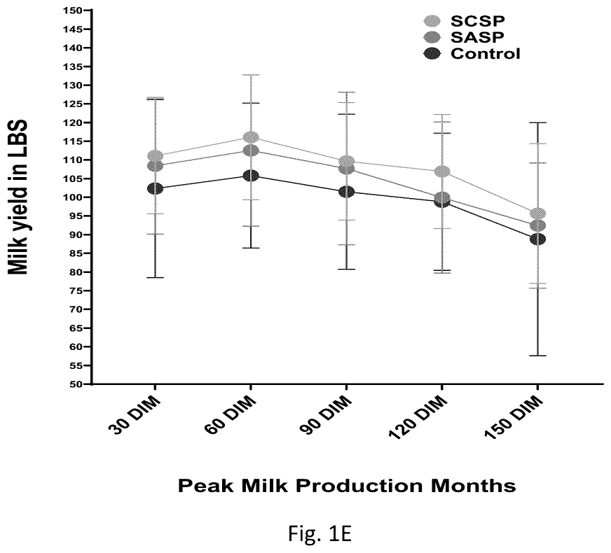

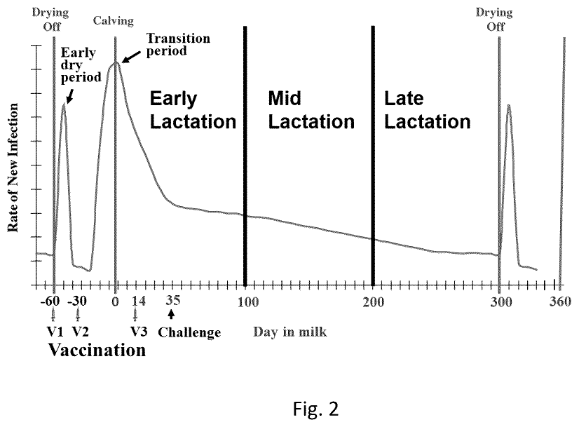

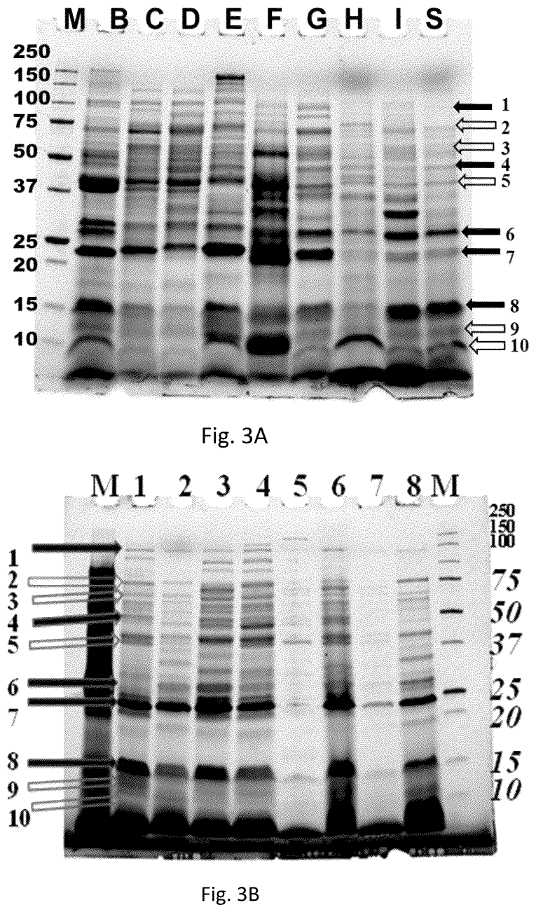

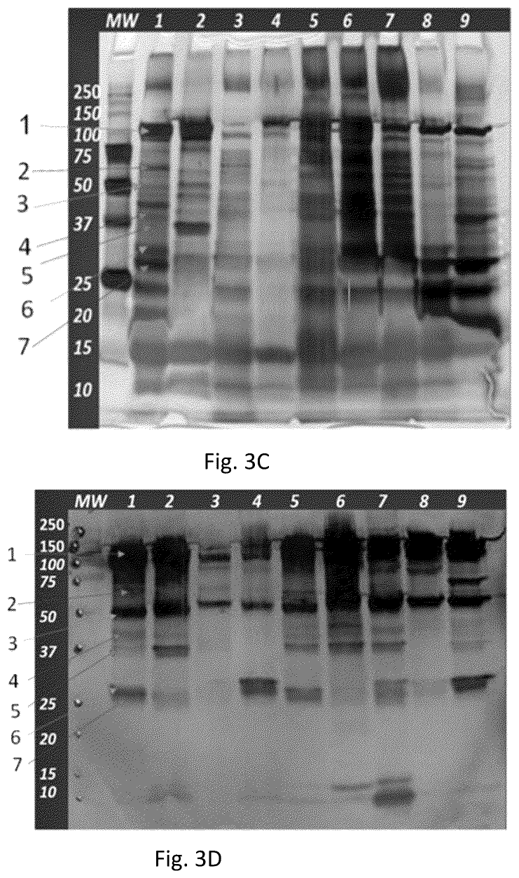

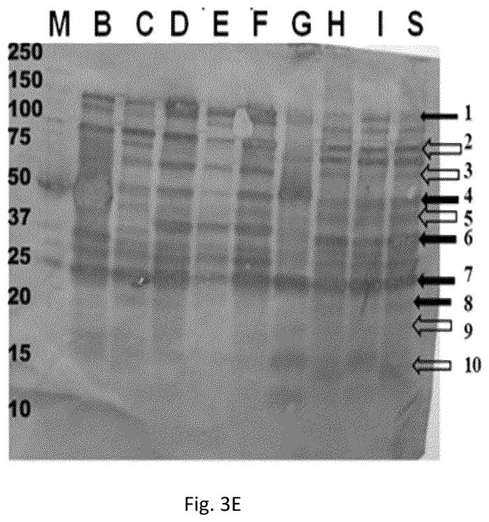

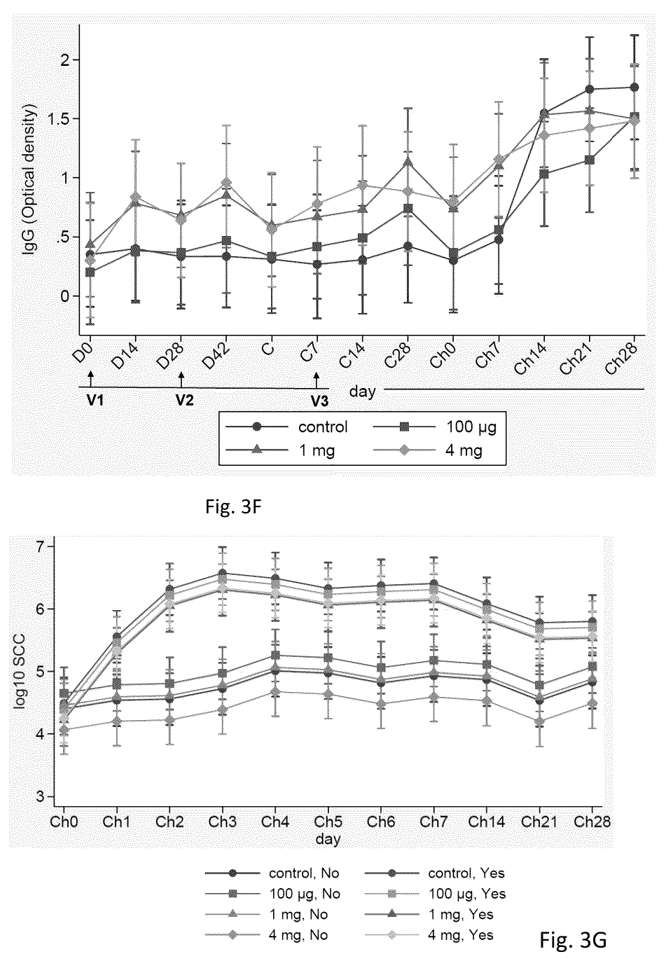

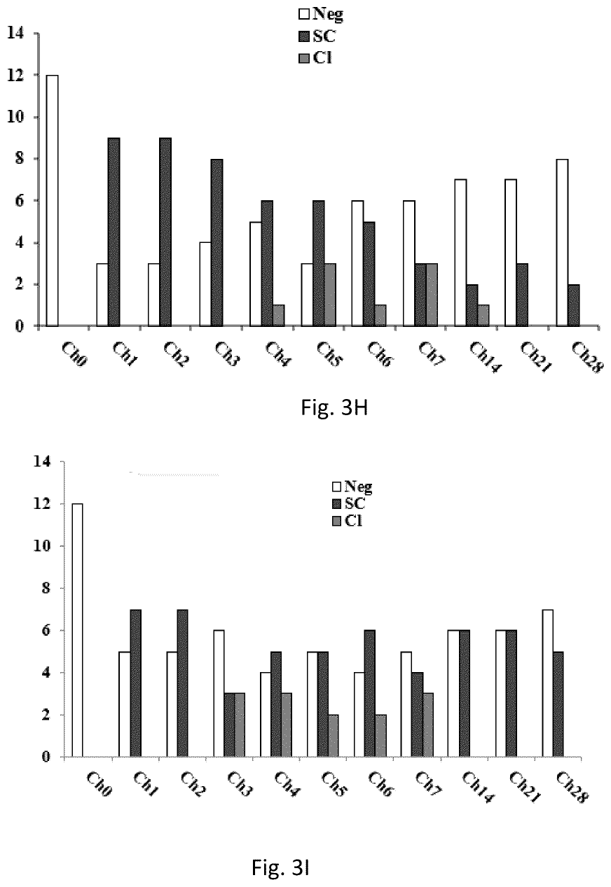

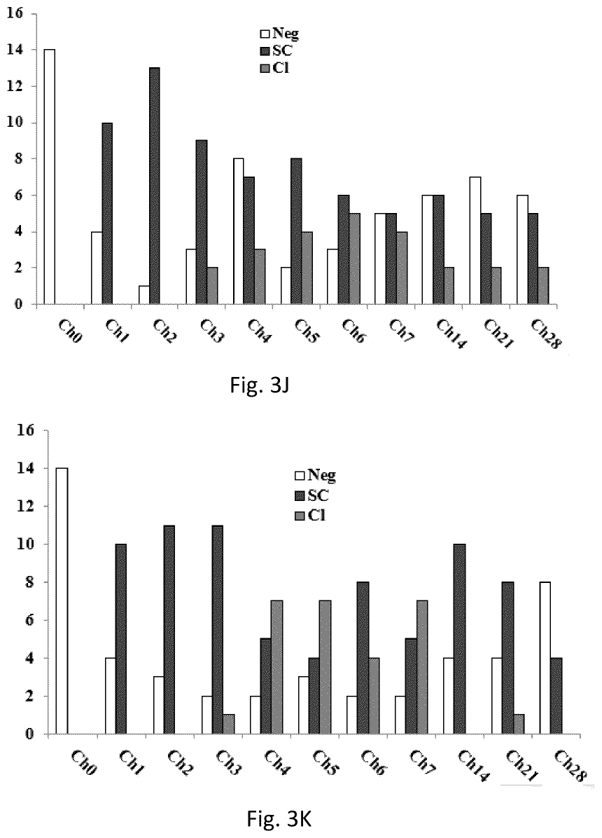

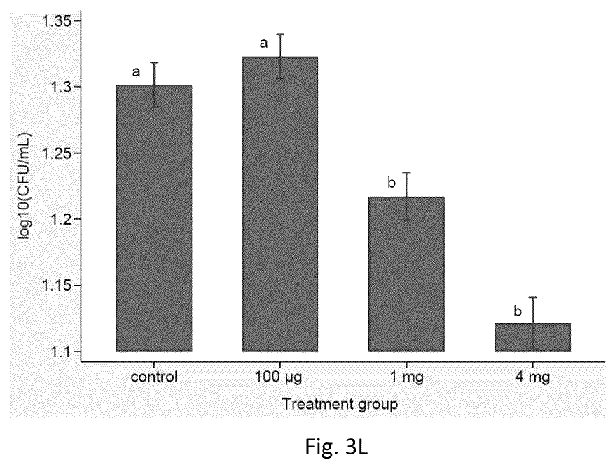

### DETAILED DESCRIPTION

### Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710) and other similar references. As used herein, the singular forms “a,” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.” As used herein, the term “comprises” means “includes.”

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. In case of conflict, the present specification, including explanations of terms, will control. In addition, the materials, methods, and examples are illustrative only and not intended to be limiting.

To facilitate review of the various embodiments of this disclosure, the following explanations of specific terms are provided:

Administration: The introduction of a composition into a subject by a chosen route. For example, if the chosen route is subcutaneous, the composition is administered by introducing the composition under the skin. If the chosen route is intravenous, the composition is administered by introducing the composition into a vein of the subject. In some examples a chemotherapeutic is administered to a subject. In some examples, disclosed peptides are administered to a subject.

Animal: Living multi-cellular vertebrate organisms, a category that includes, for example, mammals and birds. The term mammal includes both human and non-human mammals. Similarly, the term “subject” includes both human and veterinary subjects. A veterinary subject may include a dairy producing mammal. Dairy producing mammals include, but are not limited to dairy cows, sheep, goats, camels, buffaloes, yaks, horses, reindeer and donkeys. In some examples a subject is a subject, such as a subject suffering from an intramammary infection (IMI), such as a bacterial, viral, fungal or algal infection.

Effective amount or Therapeutically effective amount: The amount of agent, such as a an antiviral agent, that is sufficient to prevent, treat (including prophylaxis), reduce and/or ameliorate the symptoms and/or underlying causes of any of a disorder or disease, for example to prevent, inhibit, and/or treat an intramammary infection. In some embodiments, an “effective amount” is sufficient to reduce or eliminate a symptom of a disease, such as an intramammary infection. Reduction and/or amelioration of an intramammary infection includes, but is not limited to, a reduction in the incidence of whole cow (whole subject) disease. It is recognized that a reduction/amelioration encompasses a reduction in the incidence of whole subject disease even when the incidence of subclinical disease or quarter level disease remains the same or may even increase. Reduction and/or amelioration further encompasses a decrease in clinical level presentation, regardless of the impact on subclinical indicators.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et ah, Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Inhibit: To reduce by a measurable degree.

Inhibiting or treating a disease: Inhibiting the full development of a disease or condition, for example, for example an intramammary infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology. Inhibition of an intramammary infection may include but is not limited to a decreased rate of clinical mastitis, a decreased risk of subclinical mastitis, a decreased risk of subclinical mastitis at the quarter level, a decreased risk of subclinical mastitis at the whole subject level, and a reduction in somatic cell count (SCC).

Isolated: An “isolated” biological component, such as a peptide (for example one or more of the peptides disclosed herein), cell, nucleic acid, or serum samples has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5 ‘-end; the left-hand direction of a double-stranded nucleotide sequence is referred to as the 5’-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors, such as adenoviral vectors, comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well. A first sequence is an “antisense” with respect to a second sequence if a polynucleotide whose sequence is the first sequence specifically hybridizes with a polynucleotide whose sequence is the second sequence.

Pharmaceutically acceptable carriers: The pharmaceutically acceptable carriers of use are conventional. Remington's Pharmaceutical Sciences, by E. W. Martin, Mack Publishing Co., Easton, PA, 19th Edition (1995), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced. In some examples, a peptide is one or more of the peptides disclosed herein.

Purified: The term “purified” does not require absolute purity; rather, it is intended as a relative term. Thus, for example, a purified protein preparation is one in which the protein referred to is more pure than the protein in its natural environment within or on a cell or within a production reaction chamber (as appropriate).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2:482, 1981; Needleman & Wunsch J. Mol. Biol. 48:443, 1970; Pearson & Lipman Proc. Natl. Acad. Sci. USA 85:2444, 1988; Higgins & Sharp Gene 73:237-244, 1988; Higgins & Sharp CABIOS 5:151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutical agent: A chemical compound or composition capable of inducing a desired therapeutic or prophylactic effect when properly administered to a subject or a cell. An “anti-bacterial agent” is an agent that inhibits a bacteria from replicating or infecting cells” An “anti-viral agent” is an agent that specifically inhibits a virus from replicating or infecting cells.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses. The term vector includes plasmids, linear nucleic acid molecules, and as described throughout adenovirus vectors and adenoviruses. Virus: Microscopic infectious organism that reproduces inside living cells. A virus consists essentially of a core of nucleic acid surrounded by a protein coat, and has the ability to replicate only inside a living cell, for example as a viral infection. “Viral replication” is the production of additional virus by the occurrence of at least one viral life cycle. A virus, for example during a viral infection, may subvert the host cells' normal functions, causing the cell to behave in a manner determined by the virus. For example, a viral infection may result in a cell producing a cytokine, or responding to a cytokine, when the uninfected cell does not normally do so. Many viruses (e.g. influenza and many animal viruses) have viral envelopes covering their protein capsids. The envelopes typically are derived from portions of the host cell membranes (phospholipids and proteins), but include some viral glycoproteins. Functionally, viral envelopes are used to help viruses enter host cells. Glycoproteins on the surface of the envelope serve to identify and bind to receptor sites on the host's membrane. The viral envelope then fuses with the host's membrane, allowing the capsid and viral genome to enter and infect the host.

The goal of developing an effective staphylococcal vaccine is not to prevent IMI but to prevent clinical disease and production losses. Our lab previously developed Staphylococcus aureus surface-associated proteins (SASP) and Staphylococcus chromogenes surface-associated proteins (SCSP) vaccines against S. aureus mastitis (Merrill et al., 2019). Some of the highly immunogenic proteins in the SASP (Abdi et al., 2019) are known as cytosolic proteins but shown to be associated with the cell surface and have multifunctional roles (Henderson and Martin, 2011, Ebner et al., 2015, Widjaja et al., 2017, Harvey et al., 2019, Li et al., 2022, Du et al., 2023). Initial host-S. uberis interactions induce the expression of virulence-associated immunoreactive surface proteins (Kerro Dego et al., 2018). Based on this finding, we believe that control of S. uberis mastitis could be achieved using S. uberis surface-associated proteins (SUSP) as a vaccine to enhance the intramammary immunity more effectively than a single protein (subunit vaccine).

A previous commercial polyvalent bacterin vaccine decreased inflammatory response in vaccinated cows compared to the control cows upon experimental challenge infection (Piepers et al., 2017). However, the observed protection mechanisms for this vaccine are unknown. In contrast to the Piepers et al. study, the current study did not find a significant difference in the serum anti-SCSP and anti-SASP IgG1 titers (Th2 immune response) between vaccinated and control groups. However, the serum anti-SCSP and anti-SASP IgG2 titers (Th1 immune response) in vaccinated cows were significantly higher than that of the control cows.

Vaccination of dairy cows with two surface-associated proteins vaccines, SASP and SCSP, during the dry period and boosted during lactation induced a significantly increased SASP- and

SCSP-specific antibody titers in the serum and milk of vaccinated cows during lactation compared to the control. Subsequent monitoring of vaccinated and unvaccinated control cows for naturally occurring mastitis due to staphylococci over 300 days of lactation showed that SCSP and SASP vaccines significantly reduced the incidence of subclinical staphylococcal mastitis at the quarter level, whereas the SCSP vaccine significantly reduced the incidence of subclinical mastitis at cow level. The duration of immunity was about four months. The SCSP vaccine conferred more protection than SASP vaccine.

### Pharmaceutical Compositions

A disclosed surface-associated polypeptide or mixture of surface-associated polypeptides can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, PA, 1995) either locally or systemically, such as by subcutaneous, intramuscular, intramammarian, intraperitoneal or intravenous injection, but even oral, nasal, transdermal or anal administration is contemplated. In one embodiment, administration is by subcutaneous or intramuscular injection. To extend the time during which the peptide or protein is available, the peptide or protein can be provided as an implant, an oily injection, or as a particulate system. The particulate system can be a microparticle, a microcapsule, a microsphere, a nanocapsule, or similar particle. (see, e.g., Banga, supra).

Surface-associated polypeptides can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, PA, 1995) either locally or systemically, such as by intramuscular, subcutaneous, intramammarian, intraperitoneal or intravenous injection, but even oral, nasal, transdermal or anal administration is contemplated. In one embodiment, administration is by subcutaneous or intramuscular injection or transdermal application. To extend the time during which the peptide or protein is available, the peptide or protein can be provided as an implant, an oily injection, or as a particulate system. The particulate system can be a microparticle, a microcapsule, a microsphere, a nanocapsule, or similar particle. (see, e.g., Banga, supra).

Also disclosed are nucleic acid molecules encoding surface-associated polypeptides. In some embodiments, the nucleic acid molecules include a nucleic acid sequence encoding an amino acid sequence at least 95% identical to the amino acid sequence of surface-associated polypeptide described herein. These polynucleotides include DNA, cDNA and RNA sequences that encode the polypeptide of interest. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (e.g., L. Stryer, 1988, Biochemistry, 3.sup.rd Edition, W.H. 5 Freeman and Co., NY).

A nucleic acid encoding a surface associated polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

In the context of the compositions and methods described herein, a nucleic acid sequence that encodes a surface-associated polypeptide, is incorporated into a vector capable of expression in a host cell, using established molecular biology procedures. For example nucleic acids can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

Exemplary procedures sufficient to guide one of ordinary skill in the art through the production of vector capable of expression in a host cell that includes a polynucleotide sequence that encodes a surface associated polypeptide can be found for example in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

The disclosed surface-associated polypeptides and nucleic acids encoding the same can be administered in vitro, ex vivo or in vivo to a cell or subject. Generally, it is desirable to prepare the compositions as pharmaceutical compositions appropriate for the intended application. Accordingly, methods for making a medicament or pharmaceutical composition containing the polypeptides, nucleic acids, adenovirus vectors or adenoviruses described above are included herein. Typically, preparation of a pharmaceutical composition (medicament) entails preparing a pharmaceutical composition that is essentially free of pyrogens, as well as any other impurities that could be harmful to humans or animals. Typically, the pharmaceutical composition contains appropriate salts and buffers to render the components of the composition stable and allow for uptake of nucleic acids or virus by target cells.

Therapeutic compositions can be provided as parenteral compositions, such as for injection or infusion. Such compositions are formulated generally by mixing a disclosed therapeutic agent at the desired degree of purity, in a unit dosage injectable form (solution, suspension, or emulsion), with a pharmaceutically acceptable carrier, for example one that is non toxic to recipients at the dosages and concentrations employed and is compatible with other ingredients of the formulation. In addition, a disclosed therapeutic agent can be suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. The active ingredient, optionally together with excipients, can also be in the form of a lyophilisate and can be made into a solution prior to parenteral administration by the addition of suitable solvents. Solutions such as those that are used, for example, for parenteral administration can also be used as infusion solutions.

The peptides of the present disclosure can be provided to a subject as part of a pharmaceutical composition where it is mixed with a pharmaceutically acceptable carrier. The purpose of a pharmaceutical composition is to facilitate administration of a compound to the patient. Pharmaceutical compositions can include an effective amount of the adenovirus vector or virus dispersed (for example, dissolved or suspended) in a pharmaceutically acceptable carrier or excipient. Pharmaceutically acceptable carriers and/or pharmaceutically acceptable excipients are known in the art and are described, for example, in Remington's Pharmaceutical Sciences, by E. W. Martin, Mack Publishing Co., Easton, PA, 19th Edition (1995).

The nature of the carrier will depend on the particular mode of administration being employed. For example, parenteral formulations usually contain injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch or magnesium stearate. In addition, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the pharmaceutical compositions is contemplated. Supplementary active ingredients also can be incorporated into the compositions. For example, certain pharmaceutical compositions can include surface-associated polypeptides in a buffer. Dispersions also can be prepared in glycerol, liquid polyethylene glycols, and mixtures thereof and in oils. Under ordinary conditions of storage and use, these preparations contain a preservative to prevent the growth of microorganisms.

In some cases the compositions are administered to enhance the immune response, in such applications, the pharmaceutical composition is administered in a therapeutically effective amount. A therapeutically effective amount is a quantity of a composition used to achieve a desired effect in a subject. When administered to a subject, a dosage will generally be used that will achieve target tissue concentrations (for example, in lymphocytes) that has been shown to achieve an in vitro or in vivo effect.

Administration of therapeutic compositions can be by any common route as long as the target tissue (typically, the respiratory tract) is available via that route. This includes oral, nasal, ocular, buccal, or other mucosal (such as rectal or vaginal) or topical administration. Alternatively, administration will be by orthotopic, intradermal subcutaneous, intramuscular, intraperitoneal, intramammarian or intravenous injection routes. Such pharmaceutical compositions are usually administered as pharmaceutically acceptable compositions that include physiologically acceptable carriers, buffers or other excipients.

The pharmaceutical compositions can also be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like may be used. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components of the pharmaceutical composition are adjusted according to well-known parameters.

Additional formulations are suitable for oral administration. Oral formulations can include excipients such as, pharmaceutical grades of mannitol, lactose, starch, magnesium stearate, sodium saccharine, cellulose, magnesium carbonate and the like. The compositions (medicaments) typically take the form of solutions, suspensions, aerosols or powders. Exemplary formulations can be found in U.S. Patent publication No. 20020031527, the disclosure of which is incorporated herein by reference. When the route is topical, the form may be a cream, ointment, salve or spray. Exemplary methods for intramuscular, intranasal and topical administration of the adenovirus vectors and adenoviruses described herein can be found, for example, in U.S. Pat. No. 6,716,823, which is incorporated herein by reference.

An effective amount of the pharmaceutical composition is determined based on the intended goal, for example vaccination of a human or dairy mammal. The appropriate dose will vary depending on the characteristics of the subject, for example, whether the subject is a human or non-human, the age, weight, and other health considerations pertaining to the condition or status of the subject, the mode, route of administration, and number of doses, and whether the pharmaceutical composition includes nucleic acids or viruses. Generally, the pharmaceutical compositions described herein are administered for the purpose of stimulating and/or enhancing an immune response for example, an immune response against a viral antigen.

Therapeutic compositions that include a disclosed therapeutic agent can be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, therapeutic compositions including a disclosed therapeutic agent are administered by sustained-release systems. Suitable examples of sustained-release systems include suitable polymeric materials (such as, semi-permeable polymer matrices in the form of shaped articles, for example films, or mirocapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res.15:167-277, 1981; Langer, Chem. Tech. 12:98-105, 1982, ethylene vinyl acetate (Langer et al., Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44 (2): 58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA, 1993). Numerous additional systems for controlled delivery of therapeutic proteins are known (for example, U.S. Pat. Nos. 5,055,303; 5,188,837; 4,235,871; 4,501,728; 4,837,028; 4,957,735; and 5,019,369; 5,055,303; 5,514,670; 5,413,797; 5,268,164; 5,004,697; 4,902,505; 5,506,206; 5,271,961; 5,254,342; and 5,534,496).

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

This disclosure relates to methods for inhibiting an intramammary infection in a subject. These methods include, for example, selecting a subject in whom the intramammary infection is to be inhibited and administering an effective amount of the surface-associated polypeptides or nucleic acids encoding such polypeptides. In some embodiments, the methods can include selecting a subject in need of treatment. In some embodiments, a subject who already has an intramammary infection is selected for administration of an effective amount of the disclosed polypeptides or nucleic acids encoding such polypeptides. In other embodiments, a subject who does not yet have an intramammary infection is selected for administration of an effective amount of the disclosed polypeptides or nucleic acids encoding such polypeptides.

Intramammary infection (IMI) may be caused by a variety of pathogens including, but not limited to, bacteria, viruses, algae and fungus. Pathogens involved with intramammary infection include, but are not limited to Staphylococcus aureus, Streptococcus uberis, Staphylococcus chromogenes, Escherichia coli, Klebsiella spp., non-aureus Staphylococcal species, Mycoplasma bovis, Streptococcus agalactiae, Streptococcus dysgalactiae, Corynebacterium bovis, Enterobacter aerogenes, Streptococcus sp., Pseudomonas aeruginosa, T. pyogenes, Serratia, Pasteurella, Proteus, Bacillus, Nocardia, Enterobacter, Citrobacter, Prototheca, yeast, and mycoplasma. Intramammary infections include, but are not limited to, mastitis, clinical mastitis, subclinical mastitis, quarter-level mastitis, cow-level mastitis, and gangrenous mastitis.

This disclosure relates to methods for inhibiting a bacterial infection in a subject. These methods include, for example, selecting a subject in whom the bacterial infection is to be inhibited and administering an effective amount of the surface-associated polypeptides or nucleic acids encoding such polypeptides. In some embodiments, the methods can include selecting a subject in need of treatment. In some embodiments, a subject who already has a bacterial infection is selected for administration of an effective amount of the disclosed polypeptides or nucleic acids encoding such polypeptides. In other embodiments, a subject who does not yet have a bacterial infection is selected for administration of an effective amount of the disclosed polypeptides or nucleic acids encoding such polypeptides. For example, the subject has been exposed to a bacteria that may result in a bacterial infection in the subject.

This disclosure relates to methods for inhibiting a viral infection in a subject. These methods include, for example, selecting a subject in whom the viral infection is to be inhibited and administering an effective amount of the surface-associated polypeptides or nucleic acids encoding such polypeptides. In some embodiments, the methods can include selecting a subject in need of treatment. In some embodiments, a subject who already has a viral infection is selected for administration of an effective amount of the disclosed polypeptides or nucleic acids encoding such polypeptides. In other embodiments, a subject who does not yet have a viral infection is selected for administration of an effective amount of the disclosed polypeptides or nucleic acids encoding such polypeptides. For example, the subject has been exposed to a virus that may result in a viral infection in the subject.

By “surface-associated polypeptides” is intended the polypeptides extracted from the cell surfaces of indicated strains of bacteria without substantial cell lysis and surface-associated polypeptides produced in vitro. It is contemplated that the term “surface-associated polypeptides” encompasses one or more polypeptides having differing amino acid sequences. “Surface-associated polypeptides” is intended to encompass the mixture of one or more polypeptides having differing amino acid sequences obtained from the cell surface of the indicated bacterial strain. The extraction may involve a chemical or physical release from the bacterial cells. Surface associated polypeptides produced in vitro may or may not be harvested from a cell surface. Further, “Surface-associated polypeptides” may encompass compositions in which one or more polypeptides are enriched disproportionally to the other polypeptides in the mixture. For example, “surface associated polypeptides” is intended to encompass a collective group of polypeptides obtained without further enrichment, a collective group of polypeptides after further enrichment steps which may or may not disproportionally enrich certain polypeptides, a collective group of isolated polypeptides combined together and/or a single isolated polypeptide.

In the embodiments, isolated surface-associated polypeptides may comprise or consist of 1 to 10 different polypeptides, 1 to 20 different polypeptides, 1 to 30 different polypeptides, 1 to 40 different polypeptides, 1 to 50 different polypeptides, 1 to 60 different polypeptides, 1 to 70 different polypeptides, 1 to 80 different polypeptides, 1 to 90 different polypeptides, 1 to 100 different polypeptides, 1 to 150 different polypeptides, 1 to 200 different polypeptides, 1 to 250 different polypeptides, 1 to 300 different polypeptides, 1 to 350 different polypeptides, 1 to 400 different polypeptides, 1 to 450 different polypeptides, 1 to 500 different polypeptides, 1 to 550 different polypeptides, 1 to 600 different polypeptides or 1 to more than 600 different polypeptides. Surface-associated polypeptides may comprise polypeptides having different molecular weight characteristics. It is recognized that surface associated polypeptides may be enriched for particular individual polypeptides or enriched for polypeptides of particular molecular weights.

Each different polypeptide consists of its own amino acid sequence. The amino acids forming all or a part of a disclosed surface-associated polypeptide may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids. The amino acids may be a D- or L-amino acids.

The disclosed surface-associated polypeptides may also comprise one or more modified amino acids. The modified amino acid may be a derivatized amino acid or a modified and unusual amino acid. Examples of modified and unusual amino acids include but are not limited to, 2-Aminoadipic acid (Aad), 3-Aminoadipic acid (Baad), β-Amino-propionic acid (Bala, β-alanine), 2-Aminobutyric acid (Abu, piperidinic acid), 4-Aminobutyric acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (AHyl), 3-Hydroxyproline (3Hyp), 4-Hydroxyproline (4Hyp), Isodesmosine (Ide), allo-Isoleucine (Alle), N-Methylglycine (MeGly, sarcosine), N-Methylisoleucine (Melle), 6-N-Methyllysine (MeLys), N-Methylvaline (MeVal), Norvaline (Nva), Norleucine (Nle), and Ornithine (Orn). Other examples of modified and unusual amino acids are described generally in Synthetic Peptides: A User's Guide, Second Edition, April 2002, Edited Gregory A. Grant, Oxford University Press; Hruby V J, Al-obeidi F and Kazmierski W: Biochem J 268:249-262, 1990; and Toniolo C: Int J Peptide Protein Res 35:287-300, 1990; the teachings of all of which are incorporated herein by reference.

The surface-associated polypeptides may be made by any technique known to those of skill in the art, including chemical synthesis or recombinant means using standard molecular biological techniques. The peptides may be synthesized in solution or on a solid support in accordance with conventional techniques. Various automatic synthesizers are commercially available and can be used in accordance with known protocols. (See, for example, Stewart and Young, Solid Phase Peptide Synthesis, 2d ed. Pierce Chemical Co., 1984; Tam et al., J. Am. Chem. Soc., 105:6442, 1983; Merrifield, Science, 232:341-347, 1986; and Barany and Merrifield, The Peptides, Gross and Meienhofer, eds., Academic Press, New York, pp. 1-284, 1979, each is incorporated herein by reference in its entirety.)

Alternatively, recombinant DNA technology may be employed wherein a nucleotide sequence which encodes a peptide of the invention is inserted into an expression vector, transformed or transfected into an appropriate host cell, cultivated under conditions suitable for expression, and isolating the peptide.

In certain embodiments, the peptides may be obtained by isolation or purification. Protein purification techniques involve, at one level, the homogenization and crude fractionation of cells, tissue, or organ to peptide and non-peptide fractions. Other protein purification techniques include, for example, precipitation with ammonium sulfate, polyethylene glycol (PEG), antibodies and the like, or by heat denaturation, followed by: centrifugation; chromatography steps such as ion exchange, gel filtration, reverse phase, hydroxylapatite and affinity chromatography; isoelectric focusing; gel electrophoresis, for example polyacrylamide gel electrophoresis; and combinations of these and other techniques. Exemplary methods of obtaining surface-associated polypeptides from S. aureaus, S. chromogenes and S. uberis are disclosed elsewhere herein. However, it is recognized that any method of obtaining surface-associated polypeptides from S. aureaus, S. chromogenes and S. uberis known in the art may be used to obtain the surface-associated polypeptides.

Various chromatographic techniques include but are not limited to ion-exchange chromatography, gel exclusion chromatography, affinity chromatography, immuno-affinity chromatography, and reverse phase chromatography. A particularly efficient method of purifying peptides is fast performance liquid chromatography (FPLC) or even high performance liquid chromatography (HPLC).

The order of conducting the various purification steps may be changed, for example, or certain steps may be omitted, and still result in a suitable method for the preparation of a substantially purified peptide.

The peptides may be a part of a polypeptide or protein and may be produced by biochemical or enzymatic fragmentation of the polypeptide or protein. Accordingly, the peptides of the present invention may be (a) naturally-occurring, (b) produced by chemical synthesis, (c) produced by recombinant DNA technology, (d) produced by biochemical or enzymatic fragmentation of larger molecules, (e) produced by methods resulting from a combination of methods a through d listed above, or (f) produced by any other means for producing peptides.

During chemical synthesis, the peptides may be modified at its N- or C-terminus, thereby providing for improved stability and formulation, resistance to protease degradation, and the like. Examples of modifications of amino acids include pegylation, acetylation, alkylation, formylation, amidation. Moreover, various amino acids which do not naturally occur along the chain may be introduced to improve the stability of the peptides.

Also disclosed are nucleic acid molecules encoding these surface-associated polypeptides. In some embodiments, the nucleic acid molecules include a nucleic acid sequence encoding an amino acid sequence at least 95% identical to the amino acid sequence of a surface-associated polypeptide. In the context of the compositions and methods described herein, a nucleic acid sequence that encodes at least one surface-associated polypeptide, such as described above, is incorporated into a vector capable of expression in a host cell (for example an adenoviral vector), using established molecular biology procedures. For example nucleic acids, such as cDNAs, that encode at least one surface-associated polypeptide can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

Exemplary procedures sufficient to guide one of ordinary skill in the art through the production of vector capable of expression in a host cell that includes a polynucleotide sequence that encodes at least one heparin-binding polypeptide can be found for example in Sambrook et ah, Molecular Cloning: A Laboratory Manual, 2d ed., Cold Spring Harbor Laboratory Press, 1989; Sambrook et ah, Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al, Short Protocols in Molecular Biology: A Compendium of Methods from Current Protocols in Molecular Biology, 4th ed., Wiley & Sons, 1999.

Typically, a polynucleotide sequence encoding at least one surface-associated polypeptide is operably linked to transcriptional control sequences including, for example a promoter and a polyadenylation signal. A promoter is a polynucleotide sequence recognized by the transcriptional machinery of the host cell (or introduced synthetic machinery) that is involved in the initiation of transcription. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters are isolated from mammalian genes, including the immunoglobulin heavy chain, immunoglobulin light chain, T-cell receptor, HLA DQ α and DQ β, β-interferon, interleukin-2, interleukin-2 receptor, MHC class II, HLA-DRα, β-actin, muscle creatine kinase, prealbumin (transthyretin), elastase I, metallothionein, collagenase, albumin, fetoprotein, β-globin, c-fos, c-HA-ras, insulin, neural cell adhesion molecule (NCAM), αl-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin, dendritic cell-specific promoters, such as CDI Ic, macrophage-specific promoters, such as CD68, Langerhans cell-specific promoters, such as Langerin, and promoters specific for keratinocytes, and epithelial cells of the skin and lung.

The promoter can be either inducible or constitutive. An inducible promoter is a promoter which is inactive or exhibits low activity except in the presence of an inducer substance. Examples of inducible promoters include, but are not limited to, MT II, MMTV, collagenase, stromelysin, SV40, murine MX gene, α-2-macroglobulin, MHC class I gene h-2 kb, HSP70, proliferin, tumor necrosis factor, or thyroid stimulating hormone gene promoter.

Typically, the promoter is a constitutive promoter that results in high levels of transcription upon introduction into a host cell in the absence of additional factors. Optionally, the transcription control sequences include one or more enhancer elements, which are binding recognition sites for one or more transcription factors that increase transcription above that observed for the minimal promoter alone. It may be desirable to include a polyadenylation signal to effect proper termination and polyadenylation of the gene transcript. Exemplary polyadenylation signals have been isolated from bovine growth hormone, SV40 and the herpes simplex virus thymidine kinase genes. Any of these or other polyadenylation signals can be utilized in the context of the adenovirus vectors described herein.

Hosts cells can include microbial, yeast, insect and mammalian host cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and Hela cells, CHO cells, and WI38, BHK, and COS cell lines, although cell lines may be used, such as cells designed to provide higher expression desirable glycosylation patterns, or other features. As discussed above, techniques for the transformation of yeast cells, such as polyethylene glycol transformation, protoplast transformation and gene guns are also known in the art (see Gietz and Woods Methods in Enzymology 350:87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂) method using procedures well known in the art. Alternatively, MgCl2 or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding a disclosed heparin-binding polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

The terms “Staphylococcus aureus surface-associated polypeptides”, “SASP”, “S. aureus surface-associated polypeptides”, “Staphylococcus aureus surface polypeptides” and “S. aureus surface polypeptides” may be used interchangeably herein. SASP are surface-associated polypeptides obtained from S. aureus cells.

The terms “Staphylococcus chromogenes surface-associated polypeptides”, “SCSP”, “S. chromogenes surface-associated polypeptides”, “Staphylococcus chromogenes surface polypeptides” and “S. chromogenes surface polypeptides” may be used interchangeably herein. SCSP are surface-associated polypeptides obtained from S. chromogenes cells.

The terms “Streptococcus uberis surface-associated polypeptides”, “SUSP”, “S. uberis surface-associated polypeptides”, “Streptococcus uberis surface polypeptides” and “S. uberis surface polypeptides” may be used interchangeably herein. SUSP are surface-associated polypeptides obtained from S. uberis cells.

The pharmaceutical compositions of the present application may contain at least about 0.05 mg SASP, 0.1 mg SASP, 0.2 mg SASP, 0.3 mg SASP, 0.4 mg SASP, 0.5 mg SASP, 0.6 mg SASP, 0.7 mg SASP, 0.8 mg SASP, 0.9 mg SASP, 1 mg SASP, 1.1 mg SASP, 1.2 mg SASP, 1.3 mg SASP, 1.4 mg SASP, 1.5 mg SASP, 1.6 mg SASP, 1.7 mg SASP, 1.8 mg SASP, 1.9 mg SASP, 2 mg SASP, 2.1 mg SASP, 2.2 mg SASP, 2.3 mg SASP, 2.4 mg SASP, 2.5 mg SASP, 2.6 mg SASP, 2.7 mg SASP, 2.8 mg SASP, 2.9 mg SASP, 3 mg SASP, 3.5 mg SASP, 4 mg SASP, 4.5 mg SASP, 5 mg SASP, 5.5 mg SASP, 6 mg SASP, 6.5 mg SASP, 7 mg SASP, 7.5 mg SASP, 8 mg SASP, 8.5 mg SASP, 9 mg SASP, 9.5 mg SASP, 10 mg SASP, 100 mg SASP, 200 mg SASP, 300 mg SASP, 400 mg SASP, 500 mg SASP, 600 mg SASP, 700 mg SASP, 800 mg SASP, 900 mg SASP, 1 g SASP, 2 g SASP, 3 g SASP, 4 g SASP, 5 g SASP or more isolated surface-associated polypeptides from an indicated bacterial type.

The pharmaceutical compositions of the present application may contain at least about 0.05 mg SCSP, 0.1 mg SCSP, 0.2 mg SCSP, 0.3 mg SCSP, 0.4 mg SCSP, 0.5 mg SCSP, 0.6 mg SCSP, 0.7 mg SCSP, 0.8 mg SCSP, 0.9 mg SCSP, 1 mg SCSP, 1.1 mg SCSP, 1.2 mg SCSP, 1.3 mg SCSP, 1.4 mg SCSP, 1.5 mg SCSP, 1.6 mg SCSP, 1.7 mg SCSP, 1.8 mg SCSP, 1.9 mg SCSP, 2 mg SCSP, 2.1 mg SCSP, 2.2 mg SCSP, 2.3 mg SCSP, 2.4 mg SCSP, 2.5 mg SCSP, 2.6 mg SCSP, 2.7 mg SCSP, 2.8 mg SCSP, 2.9 mg SCSP, 3 mg SCSP, 3.5 mg SCSP, 4 mg SCSP, 4.5 mg SCSP, 5 mg SCSP, 5.5 mg SCSP, 6 mg SCSP, 6.5 mg SCSP, 7 mg SCSP, 7.5 mg SCSP, 8 mg SCSP, 8.5 mg SCSP, 9 mg SCSP, 9.5 mg SCSP, 10 mg SCSP, 100 mg SCSP, 200 mg SCSP, 300 mg SCSP, 400 mg SCSP, 500 mg SCSP, 600 mg SCSP, 700 mg SCSP, 800 mg SCSP, 900 mg SCSP, 1 g SCSP, 2 g SCSP, 3 g SCSP, 4 g SCSP, 5 g SCSP or more isolated surface-associated polypeptides from an indicated bacterial type.

The pharmaceutical compositions of the present application may contain at least about −.05 mg SUSP, 0.1 mg SUSP, 0.2 mg SUSP, 0.3 mg SUSP, 0.4 mg SUSP, 0.5 mg SUSP, 0.6 mg SUSP, 0.7 mg SUSP, 0.8 mg SUSP, 0.9 mg SUSP, 1 mg SUSP, 1.1 mg SUSP, 1.2 mg SUSP, 1.3 mg SUSP, 1.4 mg SUSP, 1.5 mg SUSP, 1.6 mg SUSP, 1.7 mg SUSP, 1.8 mg SUSP, 1.9 mg SUSP, 2 mg SUSP, 2.1 mg SUSP, 2.2 mg SUSP, 2.3 mg SUSP, 2.4 mg SUSP, 2.5 mg SUSP, 2.6 mg SUSP, 2.7 mg SUSP, 2.8 mg SUSP, 2.9 mg SUSP, 3 mg SUSP, 3.5 mg SUSP, 4 mg SUSP, 4.5 mg SUSP, 5 mg SUSP, 5. 5 mg SUSP, 6 mg SUSP, 6.5 mg SUSP, 7 mg SUSP, 7.5 mg SUSP, 8 mg SUSP, 8.5 mg SUSP, 9 mg SUSP, 9.5 mg SUSP, 10 mg SUSP, 100 mg SUSP, 200 mg SUSP, 300 mg SUSP, 400 mg SUSP, 500 mg SUSP, 600 mg SUSP, 700 mg SUSP, 800 mg SUSP, 900 mg SUSP, 1 g SUSP, 2 g SUSP, 3 g SUSP, 4 g SUSP, 5 g SUSP or more isolated surface-associated polypeptides from an indicated bacterial type.

A pharmaceutical composition of the present application may comprise at least two isolated surface-associated polypeptides selected from the group consisting of S. aureus surface-associated polypeptides (SASP), Staphylococcus chromogenes surface-associated polypeptides (SCSP), Streptococcus uberis surface-associated polypeptides (SUSP). The pharmaceutical compositions of the present application may comprise S. aureus surface-associated polypeptides (SASP), Staphylococcus chromogenes surface-associated polypeptides (SCSP) and Streptococcus uberis surface-associated polypeptides (SUSP).

An adjuvant is a substance that enhances a subject's immune response to an antigen. Specific embodiments may comprise adjuvants capable of stimulating the immune response in a dairy producing mammal. Adjuvants and veterinary adjuvants are known in the art. Veterinary adjuvants include, but are not limited to Emulsigen-D®, dimethyl-dioctadecyl ammonium bromide (DDA), and Montanide ISA 61VG®.

By “dosing regimen” is intended a schedule of treatments to be administered to a subject. A dosing regimen may comprise one or more doses. The schedule of the treatments may be determined in relation to the milk production cycle, in relation to the calving cycle, in relation to the age of the subject, in relation to a combination of these factors or by any other means of determining a dosing regimen known in the art. A dosing regimen comprises at least one dose and may be in the range of at least two doses to three doses, two doses to four doses, two doses to five doses, two doses to six doses, two doses to seven doses, two doses to eight doses, two doses to nine doses, two doses to ten doses, two doses to twenty doses, two to thirty doses, two to forty doses or two to fifty doses.

Surprisingly, the antibody responses in milk and serum of SCSP and SASP vaccinated cows increased. The cumulative incidence of subclinical staphylococcus mastitis at cow level with SCSP treatment decreased and the cumulative incidence of subclinical staphylococcus mastitis at the quarter level decreased. Both SCSP & SASP induced significantly increased specific antibody titers in milk and serum. Cows exhibited significantly reduced prevalence of CM and SCM. There was reduced cumulative incidence of subclinical staphylococcal mastitis at cow and quarter levels (SCSP). Staphylococcal surface proteins especially SCSP are effective for the control of staphylococcal mastitis in dairy cows.

The duration of immunity may be in the range of about Antibody titers decreased at 120 days in milk indicating that the duration of immunity may be about 4 months.

### Examples

The following examples further illustrate the invention but should not be construed as in any way limiting its scope.

### Example 1. Preparation of Multivalent (SASP and SCSP) Vaccine Formulations

SASP and SCSP were extracted and prepared as previously described (Abdi et al., 2019, Merrill et al., 2019). The SASP vaccine strain was S. aureus strain 38 (SAUT1), which is one of the dominant strains of S. aureus [as determined by pulsed filed gel electrophoresis (PFGE)], frequently isolated from cases of bovine mastitis across East Tennessee dairy farms between 2005-2012 (Vaughn et al., 2020).

The S. chromogenes isolate that prevented mammary gland colonization by heterologous challenge strain of S. aureus 60 (SAUT2) under in vivo conditions during an experimental challenge infection model development was used (Kerro Dego et al., 2020). A total of 1.2 mg of SASP or SCSP in 1.5 mL of phosphate buffered saline (PBS) (pH 7.4)] was mixed with 1.5 mL of Emulsigen-D (Em-D) (Phibro Animal Health Corporation, Teaneck, NJ), making a total volume of 3 mL. The control vaccine was 1.5 mL of PBS (pH 7.4) mixed with 1.5 mL of Emulsigen-D, resulting in a total volume of 3 mL. Vaccines were prepared following procedures described previously (Merrill et al., 2019). Briefly, each strain was streaked on tryptic soy agar (TSA) with 5% sheep blood (Becton, Dickinson and Company, Franklin Lakes, NJ) and incubated overnight (16-18 h) at 37° C., 5% CO₂: 95% air balanced incubator. After overnight growth, three colonies of each species were suspended in 450 ml of tryptic soy broth (TSB) and grown to the mid-log phase by incubating at 37° C. with shaking at 125 rpm. Each culture was centrifuged for 10 minutes at 500×g at 4° C. then the pellet was resuspended in 30 mL of 1% cholic acid (w/v) (Sigma-Aldrich, St. Luis, MO) and incubated at room temperature for 2 h with shaking at 125 rpm. The bacterial suspension was centrifuged at 10,000×g for 30 min at 4° C., and the supernatant containing the proteins was buffer exchanged to PBS using Cetriprep Ultracel-10K YM concentrators (10 kDa cutoff) (EMD Millipore Corporation, Billerica, MA). The proteins were filter sterilized, and concentration was measured using a nanodrop spectrophotometer (ThermoFisher Scientific, Waltham, MA).

### Example 2. Study Animals & Vaccination Schedule

The sample size was calculated based on the effect size and variance derived from the control and the SCSP vaccinated groups in a previous preliminary study, considering a 95% confidence level and 80% desired power employing Epitools-Epidemiological Calculators (Sergeant, 2018).Only cows that were healthy and bacteriologically free of mastitis caused by major bacterial pathogens, such as S. aureus, S. uberis, S. agalactiae, S. dysgalactiae and coliform bacteria (E. coli and Klebsiella spp.) were enrolled in the study. Composite (pooled milk from all quarters at equal proportion) and individual quarter milk SCC of less than 200,000 cells/mL and 100,000 cells/mL of milk, respectively, were required. Initially, 509 quarters from 130 cows were screened. Eleven quarters were blind and not included in the screening. The prevalence of S. aureus among the initially screened was 6.92% (9/130) and 1.96% (10/509) at cow and quarter levels, respectively. At cow and quarter levels, NAS culture positive milk samples were 79.23% (103/130) and 42.83% (218/509), respectively. Consequently, all S. aureus and NAS culture positive cows were excluded, and only culture negative were enrolled in the study. Cows were screened weekly starting at four weeks prior to drying off at 28, 21, 14, and 7 days before drying off. A total of 45 pregnant Holstein dairy cows in their 1^{st}-6^{th} lactation (with a median parity of 3 in the SASP and SCSP and 2 in control; P=0.5755) from the East Tennessee AgResearch and Education Center Little River Animal and Environmental Unit (ETREC-LRAEU) Dairy herd were assigned to three groups of 15 (SASP), 16 (SCSP) and 14 (unvaccinated control) cows. Animals were enrolled in the study on a continuous basis as eligible cows became available. Cows were assigned based on their coded animal IDs, and the treatment groups were also color coded to blind the person assigning the cows. Cows in the SASP and SCSP groups were vaccinated with 1.2 mg of SASP or SCSP vaccines, respectively, with Em-D adjuvant at 60, 40, and 20 days before the expected calving date (C-60, C-40, and C-20). The booster vaccinations were given at 120 and 240 days in milk (DIM). Control cows were injected with PBS (pH 7.4) mixed with Em-D at similar time points. All cows were vaccinated subcutaneously (SC) in the neck area.

A series of three subcutaneous vaccinations were given on alternate sides of the neck, roughly halfway between the top of the shoulder and the base of the ear. Injections were given utilizing sterile disposable needles and syringes. The first vaccination was given on the upper left side of the neck, the second on the right side, and the third on the lower left side of the neck. The fourth and fifth booster vaccinations were given on the lower right and upper left side of the neck. Vaccine groups were masked to all personnel administering the vaccinations and those conducting the laboratory assays. Treatments were revealed after the completion of data collection and sample analyses.

Cows were housed at the ETREC-LRAEU dairy herd facility in Walland, TN, under the same herd care for the duration of the study. Cows were housed in free-stalls containing loose-pack sand bedding, with management adding new bedding weekly. The study cows were kept with other cows on the farm under similar management. They were usually fed a mixed total ration and other supplements required for dairy cows following the normal standard operating protocol of the farm. This study was approved by the University of Tennessee Institutional Animal Care and Use Committee (Registration Number: 2655-1118).

### Example 3. Vaccine Safety Monitoring

Vaccine safety was evaluated by monitoring rectal temperature and injection site reaction volume post-vaccination to ensure the reactions were all within normal limits.

Cows were monitored throughout the study for any adverse reactions to vaccines by measuring rectal temperature. Vaccine injection site reaction was measured in volume by taking three-dimensional measurements of height (depth), width (dorsal/ventral), and length (cranial/caudal) in millimeters (mm). The standard ellipsoid volume (V=π*L*W*H)/6 was calculated in cubic centimeters (Powers et al., 2007). Measurements were taken immediately prior to vaccination, daily for three consecutive days after each vaccine injection, on days 7 and 14 post injection, and monthly between vaccinations. Cows were monitored daily by the research team member, who is also ETREC-LRAEU staff, for any signs of mastitis and other diseases and associated abnormal behavioral manifestations, including lethargy, loss of appetite, decreased or loss of milk production, or any other complications.

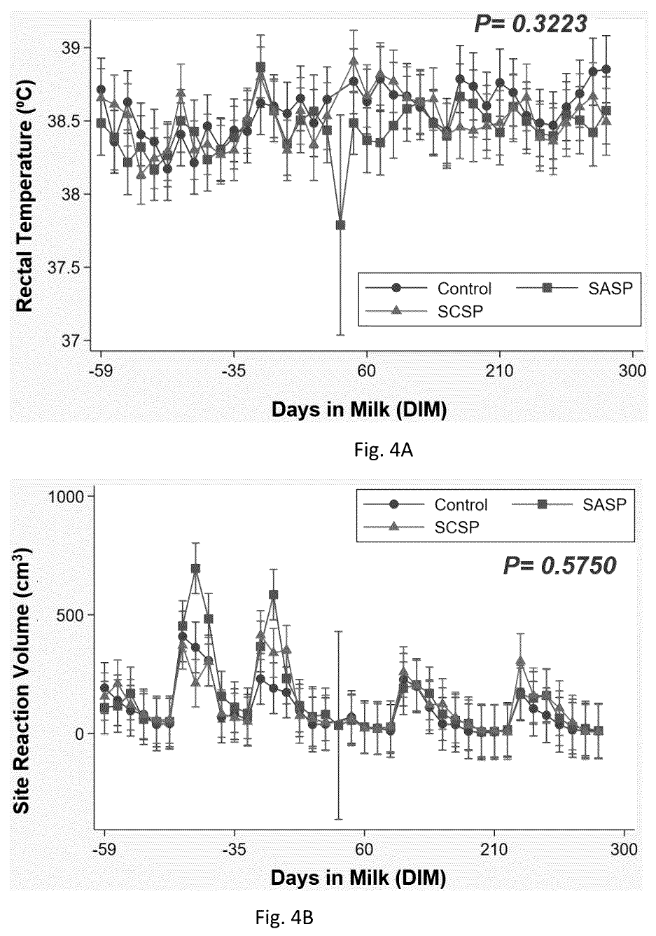

Results of rectal temperature monitoring and vaccine injection site reaction volumes from one experiment are summarized in FIGS. 4A and 4B. In the experiment shown, mean rectal temperature did not significantly vary by vaccination (P=0.0727); however, day had a significant (P<0.0001) effect. Mean rectal temperature did not significantly (P=0.667) vary by the group between the first three consecutive days (acute phase) and 7 and 14 days (delayed phase) after vaccine injection. Both time periods (P<0.0001) and their interactions with vaccination (P=0.0331) were significantly associated with the mean reaction size at the sites of injections, with no main effect of vaccination (P=0.5750). Both mean rectal temperature and sizes of the injection site reactions temporarily increased immediately (i.e., during the acute phase) following the primary (1st-3rd) and booster (4th and 5th) vaccinations, after which it declined during the delayed phase.

While there were no statistically significant differences between vaccinated and unvaccinated control groups in either the rectal temperature or the site reaction volume, there were slight increases in both categories after each vaccination. The slight increase in rectal temperatures and vaccine injection site reaction volumes in the vaccinated group were in line with previous observations utilizing the same vaccines.

### Example 4. Effects of Vaccinations on Serum Anti-SASP and Anti-SCSP Antibody Titers

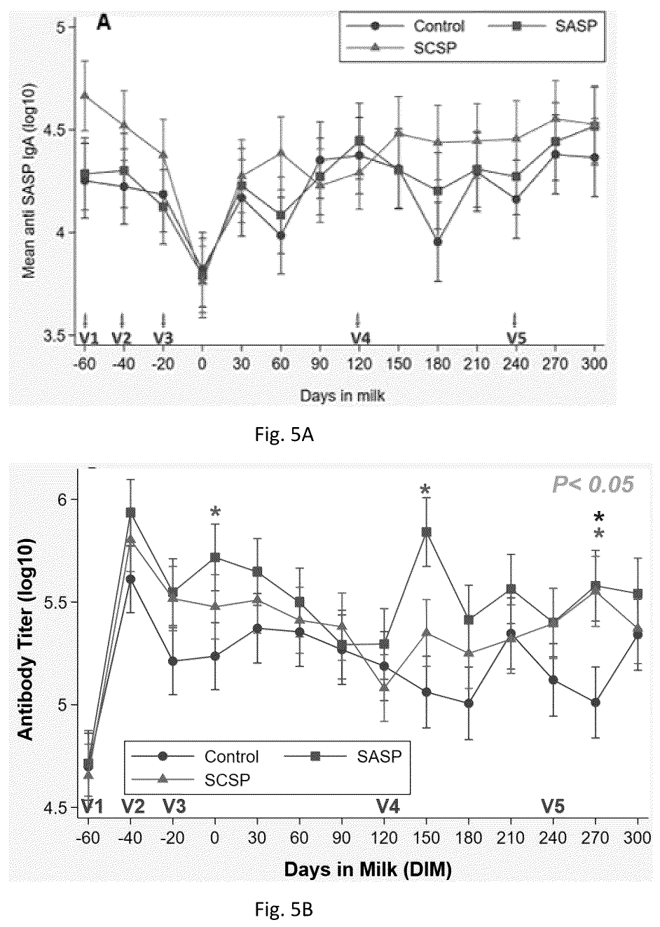

In one experiment, vaccination of dairy cows with SASP or SCSP had a significant effect on serum anti-SASP IgA (P=0.0012), IgG1 (P=0.0465), and IgG2 (P<0.0001) titers. SASP vaccinated cows had significantly higher serum anti-SASP IgG titers at calving (0 DIM, P=0.037), 150 (P<0.0001), and 270 (P=0.004) DIM; IgG1 titers at 150 DIM (P=0.012); IgG2 titers at −20 (P=0.028), 0 (P=0.005) and 150 (P=0.002) DIM compared to the control cows. SASP vaccinated cows had significantly higher cross-reacting serum anti-SCSP IgG titers at 270 DIM (P=0.011) compared to the control cows. See for example FIGS. 5A-5D.

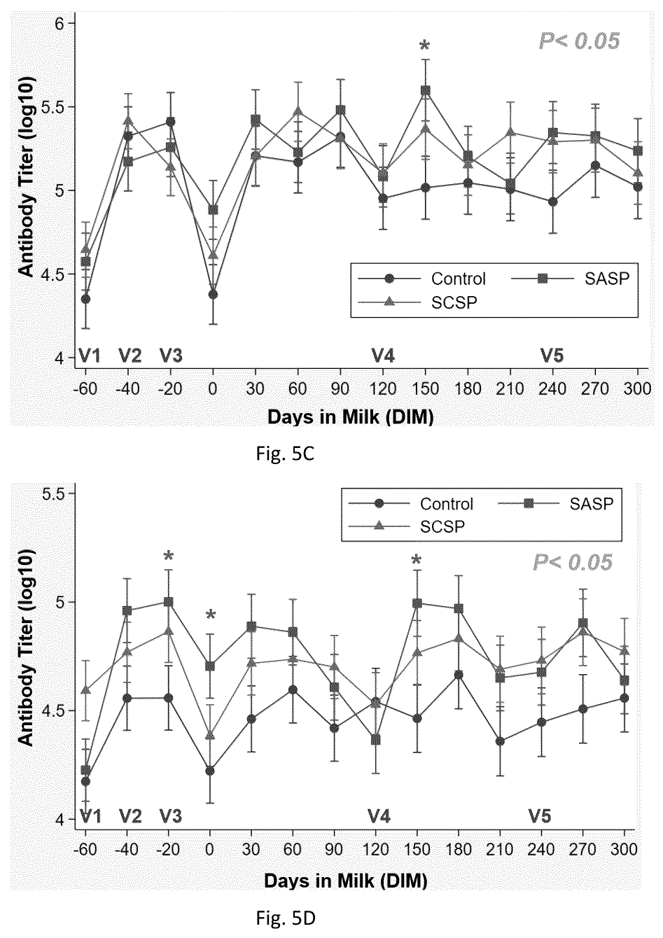

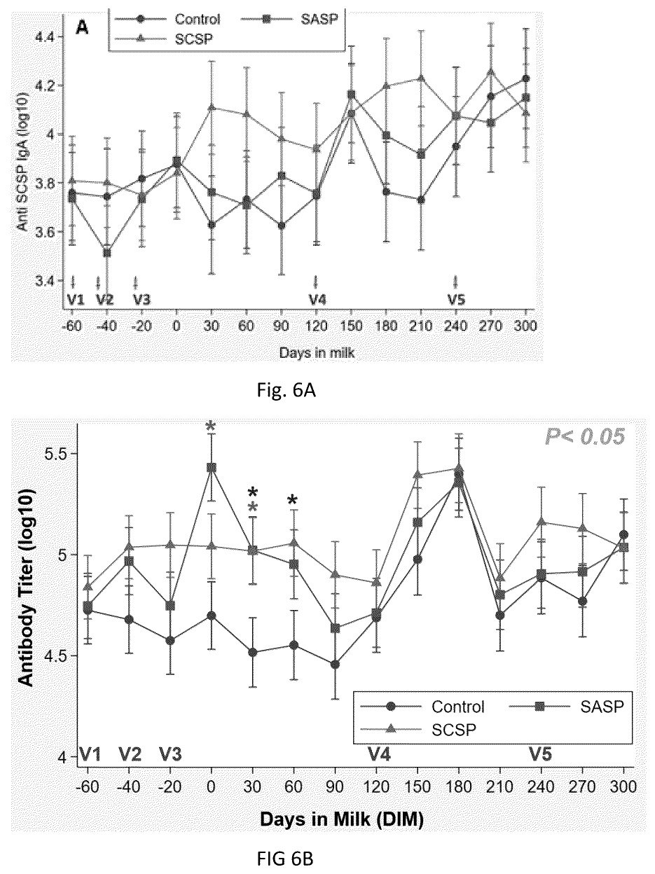

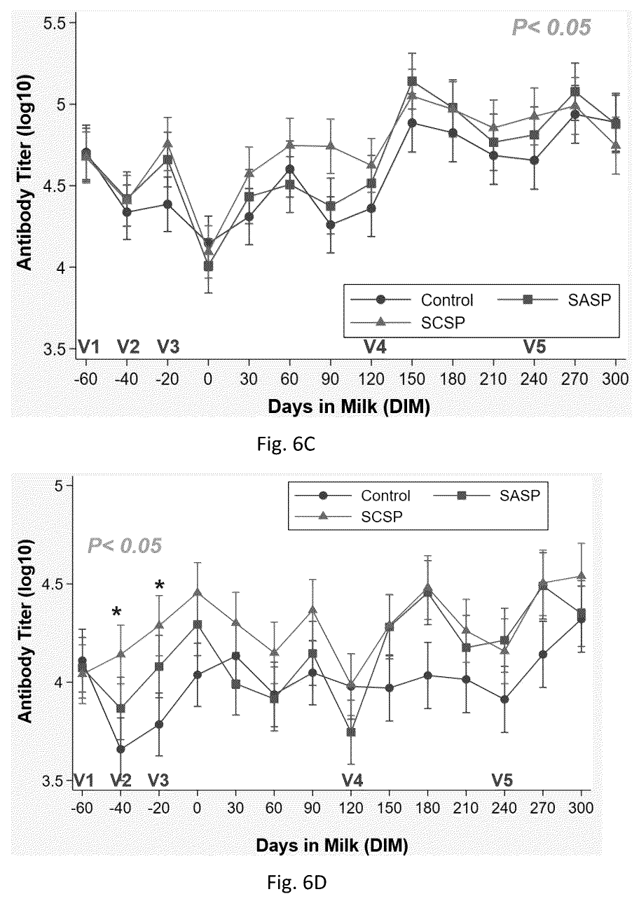

In one experiment, SCSP vaccinated cows had significantly higher serum anti-SCSP IgG titers at 30 (P=0.032) and 60 (P=0.026) DIM; IgG2 titers at −40 (P=0.013) and −20 (P=0.007) DIM compared to the control cows. The SCSP vaccinated cows had significantly higher cross-reacting serum anti-SASP IgG titers at 0 (P<0.0001) and 30 (P=0.029) DIM compared to the control cows (FIG. 6A-6D). Vaccination of dairy cows with SASP or SCSP did not significantly affect the mean serum anti-SASP and anti-SCSP IgA titers (P=0.8593) (FIGS. 5A and 6A).

### Example 5. Effects of Vaccinations on Milk Anti-SASP and Anti-SCSP Antibody Titers

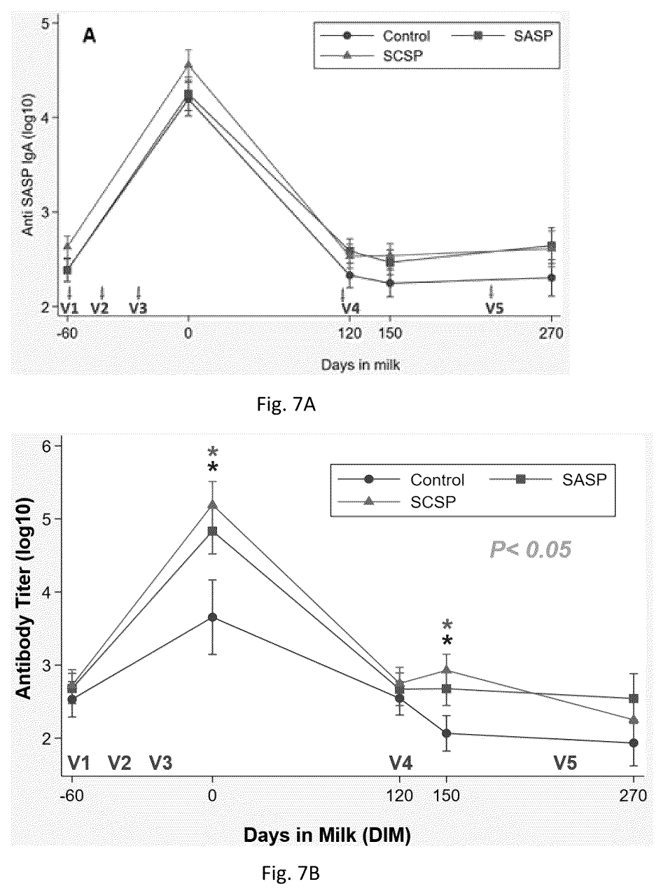

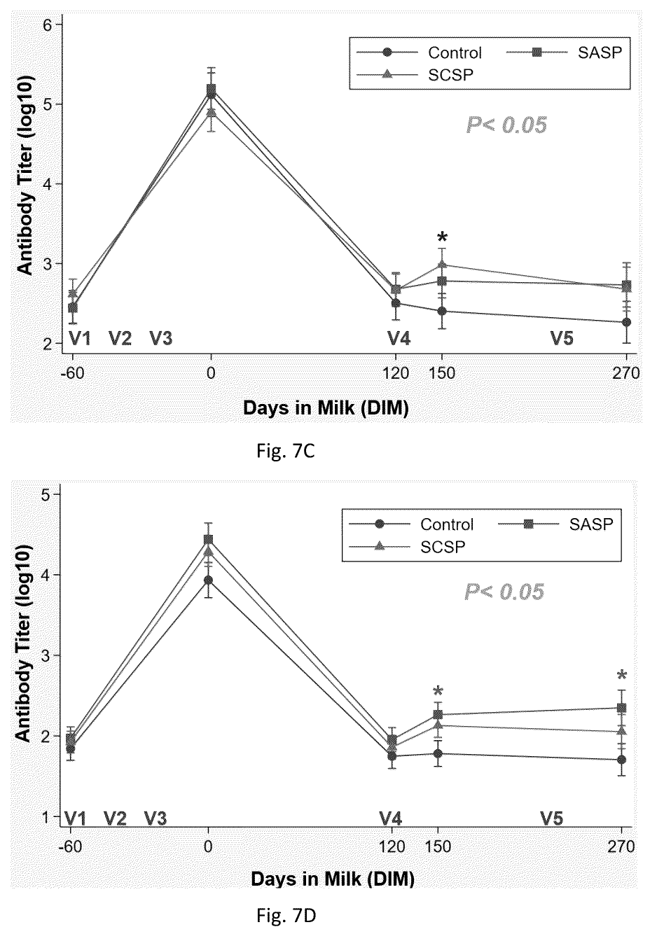

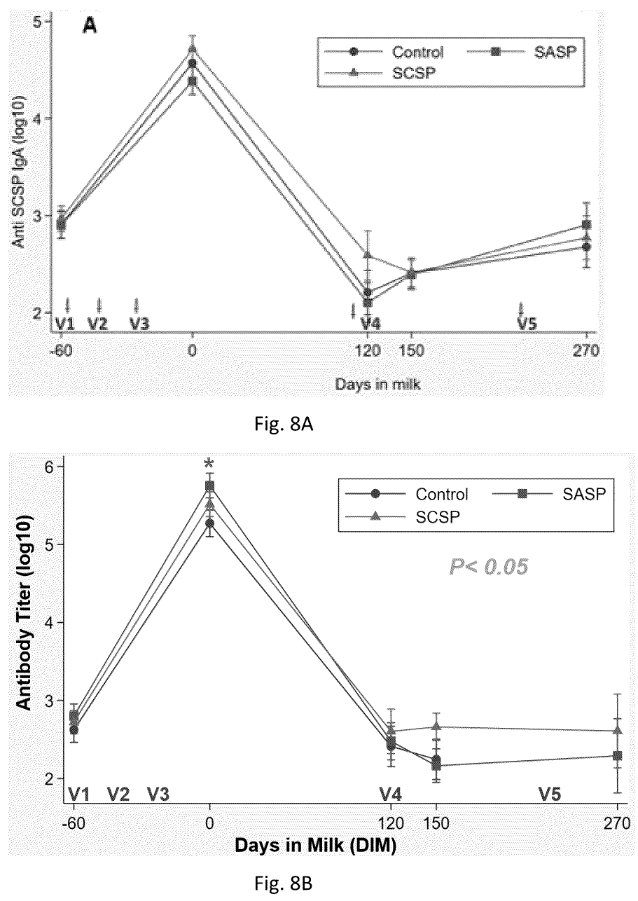

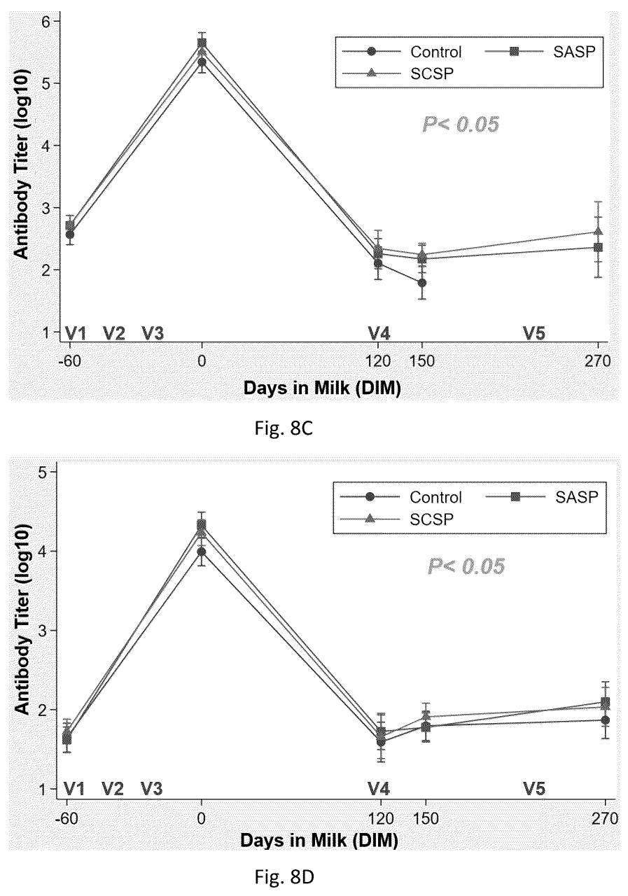

In one experiment, SASP vaccinated cows had significantly higher milk anti-SASP IgG titers at 0 (P=0.012) and 150 (P=0.037) DIM; IgG2 titers at 150 (P=0.002) and 270 (P=0.002) DIM compared to control cows. SASP vaccinated cows had significantly higher cross-reacting milk anti-SCSP IgG titers at 0 (P<0.0001) and 150 (P<0.0001) DIM; IgG1 titers at 150 DIM (P=0.018) compared to control cows (FIGS. 7A-7D). SCSP vaccinated cows had significantly higher cross-reacting milk anti-SASP IgG titers at 0 DIM (P=0.005) compared to the control cows. SCSP vaccinated cows did not have significantly different milk anti-SCSP titers compared to control cows (FIGS. 8A-8D).

Vaccination of dairy cows with SCSP or SASP vaccines induced significantly increased anti-SCSP and anti-SASP antibody titers both in the milk and serum of vaccinated cows compared to unvaccinated control cows. These results are similar to our previous findings based on a challenge study during the dry period where vaccination of dairy cows with SASP and SCSP vaccines induced significantly increased SASP-specific serum IgG2 and SCSP-specific serum IgG1 titers at post-vaccination days (Merrill et al., 2019). The current study showed that the SCSP vaccine conferred better protection than the SASP vaccine and control group under natural exposure observed over the entire lactation cycle. The incidence rate of staphylococcal mastitis was significantly reduced by SCSP and SASP vaccines at the quarter level and only by SCSP vaccine at the cow level compared to the control group. However, detailed comparative evaluation results showed that in the current study, SASP- and SCSP-specific IgG, IgG1, IgG2, and IgA titers in the serum and milk were higher than that of control at almost all time points throughout the 300 days of lactation but achieved statistical significance only at few time points. The overall higher titers at almost all time points in the vaccinated groups compared to the unvaccinated control group indicate good immunogenicity of SASP and SCSP vaccines. The SCSP vaccine significantly reduced the rate of subclinical staphylococcal mastitis as compared to the rate of subclinical staphylococcal mastitis the control at the cow level. The rate at which staphylococcal subclinical mastitis develops at a quarter level was significantly slower in the SCSP and SASP vaccinated group compared to the control.

In a previous study (Merrill et al., 2019), because of IACUC enforced strict protocol, experimental challenge infection and efficacy evaluation were conducted over a shorter period of 14 days, whereas in the current study, cows were monitored over 300 days of lactation. Surprisingly, some protective effect indicators (mastitis occurrence, staphylococcal bacteria presence in milk, SCC, milk production) over 300 days of lactation are found in cows treated with SASP or SCSP. The previous study was conducted during the dry period (Merrill et al., 2019), whereas the current study was over the lactation period of 300 days.

Unlike most previous efficacy studies, our results showed that the SCSP vaccine significantly reduced the incidence of subclinical staphylococcal mastitis when analyzed both at cow and quarter levels. There is an observed protective effect amongst the vaccinated groups compared to the control group, especially with the SCSP vaccine. The IgG2 immune response, which is Th1 associated, was robust and higher in serum and milk samples.

### Example 6. Detection of Staphylococcus aureus, Non-Aureus Staphylococci and Other Bacteria

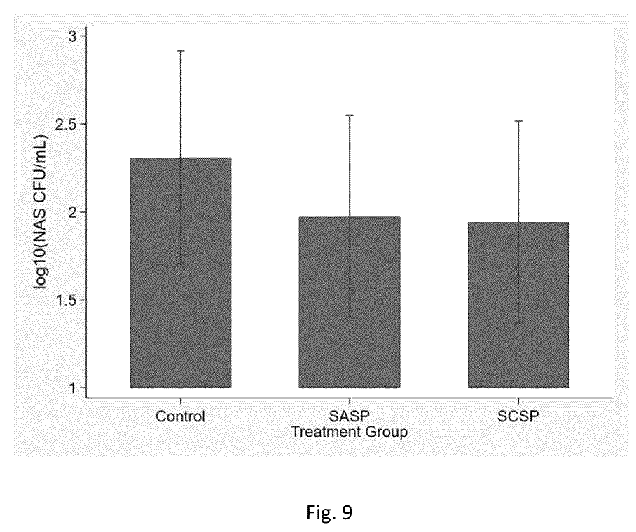

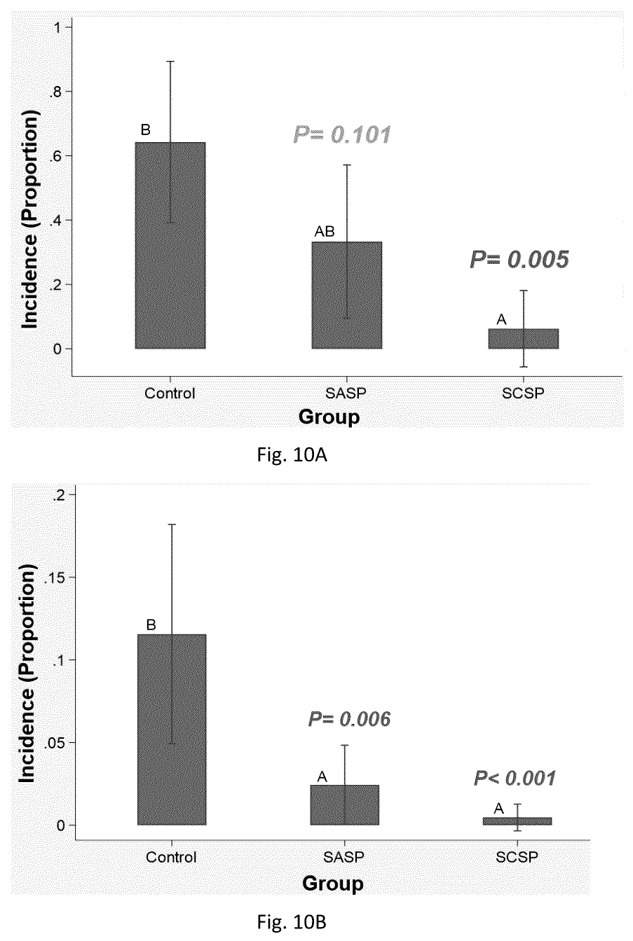

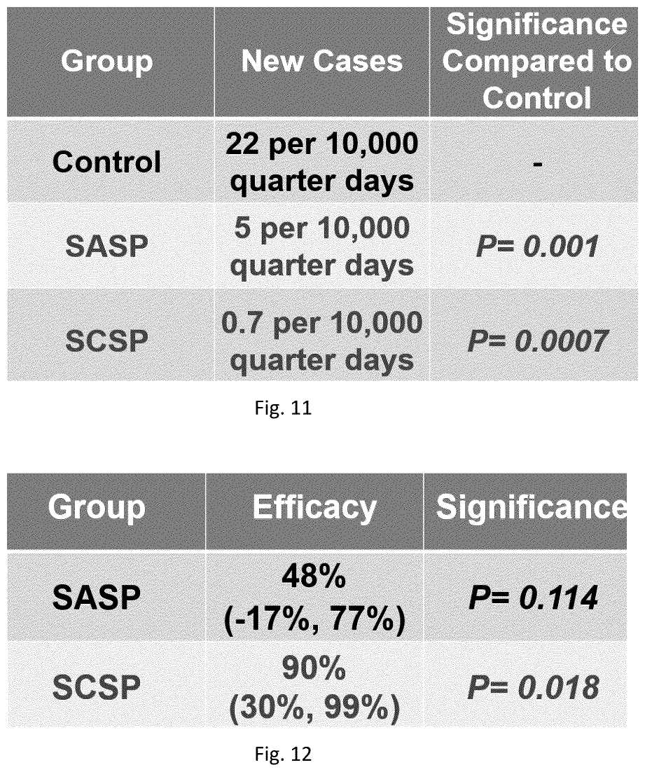

In one experiment, non-aureus staphylococci (NAS) were detected from 18.6% (n=2,025) of quarter milk samples tested during the study time of 300 days of lactation across the three vaccine groups. A total of 377 NAS isolates were identified from individual quarter samples (Control: n=133; SASP: n=129; SCSP: n=115). While the main effects of vaccination (P=0.5825) and day (P=0.4800) did not have any significant effects, vaccination-by-day interaction had a significant (P=0.0305) effect on the mean NAS count (FIG. 9). Staphylococcus aureus was isolated only from five quarters of four cows (two each from control and SASP groups). One of the control cows had 20 CFU/mL in the two front quarters on 300 DIM; the other cow had 80 CFU/mL in the left front quarter on 180 DIM. Two cows in the SASP group had 50 and 150 CFU/mL, respectively, in their right front quarters at calving.

Bacteria other than staphylococci were also isolated and identified from a few individual quarter milk samples (Table 1). Overall, there was an even distribution of the identified bacteria across groups.

TABLE 1
The number of individual quarter milk samples positive
for bacteria other than Staphylococcus species

| | | Gram | Gram | | Coryne- | | |
|---|---|---|---|---|---|---|---|
| | Bacillus | Negative | Positive | Strep. | bacterium | | |
| Group | spp. | Rod | Rod | spp. | spp. | Mold | Yeast |
| SASP | 78 | 12 | 1 | 32 | 5 | 3 | 5 |
| SCSP | 74 | 12 | 1 | 27 | 10 | 3 | 16 |
| Control | 64 | 13 | 1 | 21 | 6 | 1 | 5 |

SASP: Staphylococcus aureus surface associated proteins;
SCSP: Staphylococcus chromogenes surface associated proteins

### Example 7. Effect of Vaccinations on Milk Production and Somatic Cell Count

Milk production after vaccination was evaluated (total milk volume/group). Vaccination did not have a significant (P=0.9375) effect on milk production (data not shown). Somatic cell count in the milk was evaluated after vaccination. The mean log 10 SCC did not significantly differ among the vaccine groups (P=0.4393) (data not shown).

### Example 8. Evaluation of Cow Level Clinical and Subclinical Staphylococcus aureus and Non-Aureus Staphylococcal Mastitis

At the cow level, three cows developed clinical mastitis (6.6%, n=45), of which 14.2% (2/14) and 6.6% (1/15) were in the control and SASP vaccinated cows, respectively, all by S. aureus with no significant (Fisher's exact test P=0.1934) vaccination effect. None of the SCSP vaccinated cows developed clinical mastitis. Fifteen cows (33.3%; n=45) developed subclinical mastitis (SCM), of which 64.3% (9/14) in the control group, 8 by NAS and 1 by S. aureus, 33.3% (5/15) in the SASP group by NAS, and 6.3% (1/16) in the SCSP group by NAS with a significant (Fisher's exact test P=0.0031) vaccination effect. Binary logistic regression analysis showed that vaccination with SCSP significantly (P=0.005; OR=0.04: 95% CI=0.004-0.4) reduced cow-level incidence of SCM compared to the control group. However, vaccination with SASP did not have a significant (P=0.101) effect on the prevalence of SCM compared to the control group (Table 2).

TABLE 2
Summary table for comparing vaccination effects on cow-level subclinical mastitis

| | Groups | | | | | |
|---|---|---|---|---|---|---|
| Variables Measured | 1 (SASP) | | 2 (SCSP) | | 3 (Control) | |
| Absolute risk, % | 33.3 | (9.5, 57.2) | 6.3 | (0, 18.1) | 64.3 | (43.5, 95.0) |
| Odds ratio, compared to | 0.28 | (0.06, 1.3) | 0.04 | (0.004, 0.37) | 1 | |
| control | | | | | | |
| Risk difference, control- | −0.31 | (−0.66, −0.04) | −0.58 | (−0.86, −0.30) | 0 | |
| vaccine group | | | | | | |
| Risk ratio, relative to | 0.52 | (0.23, 1.17) | 0.1 | (0.01, 0.7) | 1 | |
| control | | | | | | |
| Vaccine efficacy, relative | 0.48 | (−0.17, 0.77) | 0.9 | (0.3, 0.99) | 0 | |
| to control | | | | | | |
| Incidence density ratio, | 1.3 | (0.52, 3.2) | 0.2 | (0.03, 1.6) | 3.8 | (1.9, 7.5) |

new cases/1000 cows

Risk ratio analysis showed that the SCSP vaccine significantly (P=0.018) reduced the risk of staphylococcal SCM by 10-fold, whereas that of SASP was not significant (P=0.114). The SCSP showed 90% (95% CI=30-99%) vaccine efficacy with a significant protective effect; the SASP vaccine had a vaccine efficacy of 48% (95% CI=−17-77%), although this was not statistically significant (Table 2). Only the SCSP vaccine group had a significant (P=0.0096) effect on the incidence density; SASP (P=0.057) was not significantly different from the control group (FIG. 8A). 3.8, 1.3, and 0.2 new SCM cases per 1000 cow days in the control, SASP, and SCSP groups, respectively were observed (Table 3).

TABLE 3
Incidence Density of Subclinical Mastitis at Cow Level

| Groups | Number of New Cases | Significance Compared to Control |
|---|---|---|
| Control | 3.8 per 1000 cow days | — |
| SASP | 1.3 per 1000 cow days | P = 0.057 |
| SCSP | 0.2 per 1000 cow days | P = 0.0096 |

SASP: Staphylococcus aureus surface associated proteins;
SCSP: Staphylococcus chromogenes surface associated proteins

### Example 9. Quarter-Level Clinical and Subclinical Staphylococcus aureus and Non-Aureus Staphylococcal Mastitis

Throughout the study time of 300 days in milk, clinical mastitis caused by S. aureus was observed in four quarters in the control (2 quarters) and the SASP (2 quarters) groups. The two quarter cases in the SASP group were from the same cow and were observed on different milk collection days, while the other two cases in the control group were observed during the intervals between subsequent monthly milk samplings. Overall, 85 cumulative quarter-level staphylococcal SCM incidences were observed during the study. The mixed effects logistic regression model showed that the main effect of vaccination was significantly (P=0.0005) associated with quarter-level SCM. Both SASP (P=0.006) and SCSP (P<0.001) vaccines significantly reduced the incidence of SCM compared to the control group. The cumulative incidence of SCM caused by any staphylococci was 11.6% (control), 2.4% (SASP), and 0.5% (SCSP) by group (8B). Both the SCSP (P=0.0007) and SASP (P=0.001) vaccines had a significant effect on the incidence density. New cases of SCM per 10,000 quarter days in the control, SASP, and SCSP groups were observed at 22, 4.8, and 0.7, respectively.

### Example 10. Preparation of Polyvalent (SASP, SCSP and SUSP) Vaccine Formulations

The SASP, SCSP and SUSP are extracted from vaccine strains of Staphylococcus aureus and Staphylococcus chromogenes as described previously (Abdi et al., 2019, Merrill et al., 2019). Similarly, SUSP is extracted following protocol optimized for SUSP (Kerro Dego et al., 2021). Each bacterium is streaked onto blood agar plates and incubated at 37° C. overnight. After incubation, 3 isolated colonies of staphylococcal spp. (S. aureus and S. chromogenes) are suspended in 450 ml of tryptic soy broth whereas 3 isolated colonies of Streptococcus uberis are inoculated to 450 mL of Todd Hewwit Broth (THB) and allowed to grow to mid-log phase at 37° C. in 5% CO₂: 95% air incubator. The culture is centrifuged at 5000×g for 10 min at 4° C. The pellet is resuspended in 30 mL of 1% cholic acid (Sigma-Aldrich) and incubated at room temperature for 2 h. Bacterial suspension is centrifuged at 10000×g for 30 min at 4° C. and proteins in the supernatant is concentrated with 10 kDa cut off membrane (EMD Millipore Corporation). Protein concentration is measured using a BCA protein assay kit (Thermofisher Scientific). The SASP, SCSP and SUSP proteins are buffered exchanged to PBS (pH 7.4) and filter sterilized. Two doses of polyvalent vaccine are prepared with 200 and 400 μg dose of each vaccine. The total dose of polyvalent vaccine (600 μg or 1.2 mg) in 1.5 mL of sterile endotoxin-free PBS (pH7.4) is mixed with 1.5 mL of Emulsigen-DR adjuvant (Phibro Animal Health). The Emulsigen®-D (Phibro Animal Health) adjuvant is known to stimulate humoral antibody, and the added nanoparticle adjuvant dimethyl-dioctadecyl ammonium bromide (DDA) is a known T-cell immune stimulator (Hilgers and Snippe, 1992) that allows for more rapid onset of immunity and enhanced protection. The proportion of adjuvant to antigen (vol/vol) for Emulsigen®-D will be 50/50 as recommended by the manufacturer (Phibro Animal Health Corp., Omaha, NE). An alternative adjuvant is Montanide ISA 61VG® (MIV61) (Seppic, France). This adjuvant is capable of enhancing the immune response in dairy cattle as shown by previous studies (Rainard et al., 2015, Herry et al., 2017).

### Example 11 Experimental Animals and Vaccination Schedule

A total of 60 pregnant Holstein dairy cows from East Tennessee AgResearch and Education Center Little River Animal and Environmental Unit dairy herd (ETREC-LAEU), Walland, TN, are enrolled in this study in 2 groups of 30 cows each. Animals are in overall good health, free of any complicating diseases, including Johne's, Brucellosis, Tuberculosis, and bovine leukemia virus. They will not have received any chemotherapy, systemic antibiotics or other anti-inflammatory medication prior to enrollment. Animals are in good body condition (mean body condition score of 3-4 on a 5-point scale) and free of IMI by major mastitis pathogens. Animals have a low antibody titer against SASP, SCSP and SUSP. Animals are randomized into one of 2 groups with 30 cows each. Cows in group 1 are vaccinated with 1.2 mg of polyvalent vaccine with Emulsigen-D respectively and challenged with S. aureus. Cows in group 2 are injected with PBS mixed with Emulsigen-D and serve as control group (Table 1).

TABLE 2
Proposed Vaccination Protocol for Polyvalent triple-S (SASP/SCSP/SUSP)
Vaccine with Emulsigen ®-D (Em-D) Adjuvant

| | # of | | | | Challenged at |
|---|---|---|---|---|---|
| Group | animals | Vaccine/adj | Dose/volume | Route/Frequency | 35 DIM |
| 1 | 30 | Polyvalent | 1.2 mg/3 mL | Subcutaneous/2 | Staph. aureus |
| | | triple-S/Em-D | | times at 60 & 30 | |
| 2 | 30 | PBS/Em-D | 3 mL | days before | |
| | | | | expected calving, | |
| | | | | 14 days in milk (DIM) | |

Adj = adjuvant; Em-D = Emulsigen ®-D, DIM: days in milk.

### Example 12 Vaccination and Clinical Assessment of Cows Following Vaccination

Vaccines are masked to personnel administering the vaccinations, evaluating safety and clinical scores, and conducting subsequent assays. Treatment codes are not broken until data collection and analyses are completed. Experimental cows are housed and managed similarly. All injections are done using sterile disposable syringes and sterile needles.

Animals are monitored after each vaccination as described (Merrill et al., 2019) to assess any adverse reactions to the vaccine. Briefly, Rectal temperatures are taken 24 h prior to and immediately before vaccination, daily for 3 d, and again at 7 and 14 d following each vaccination. Vaccine injection site reaction are measured in volume by taking three-dimensional measurements, height (depth), width (dorsal/ventral), and length (cranial/caudal) in millimeters (mm). The standard ellipsoid volume (V=π*L*W*H)/6 is calculated in cubic centimeter (Powers et al., 2007). Cows are monitored daily by qualified farm and laboratory personnel for any clinical signs of mastitis and other diseases and associated abnormal behavioral manifestations including lethargy, loss of appetite, decreased or loss of milk production, or any other complications. Animals that develop serious complications or develop disease are removed from the study.

### Example 13 Sample Collection

Blood-Blood samples for evaluation of humoral and cellular immune responses are collected immediately before each immunization at 60 and 30 days before expected calving date and at calving (CO), and at 14 and 35 days in milk (DIM) post calving. At the same time points, blood samples (50 mL) for analysis of adaptive immune response associated cytokine expression are collected from each animal in separate tubes with heparin for analysis of cytokines in whole blood (Whale et al., 2006).

Milk-Individual quarter milk samples for somatic cells counts (50 mL) and for bacteriological culture (3 mL) are collected immediately before vaccinations at 60, 30 days before expected calving date and at calving (CO) and at 14 and 35 DIM. Composite milk samples from all quarters at equal proportion for antibody titers analysis (50 mL) and for milk leukocytes isolation and testing of adaptive cytokine expressions (500 mL) are collected at above mentioned similar time points. Composite milk samples for isolation of mammary leukocytes are collected in sterile 500 ml bottles and kept on ice until cells are isolated from the milk within 4 h of collection. For analysis of antibody titers, 50 ml of composite milk samples are ultra-centrifuged at 20,000×g for 30 min to remove fat and cellular debris. Following centrifugation, the casein pellet is discarded and the remaining skimmed milk is stored at −80° C. until antibody titers measured by enzyme linked immunosorbent assay (ELISA). Microbiological evaluation of milk is conducted following procedures recommended by the National Mastitis Council (Oliver et al., 2004). Somatic cells count are determined at the Dairy Herd Improvement Association Laboratory, Knoxville, TN.

### Example 14 Enzyme-Linked Immunosorbent Assay (ELISA)

This assay is conducted as described previously (Merrill et al., 2019, Kerro Dego et al., 2021). Briefly, the SASP or SCSP or SUSP are diluted and coated onto each well of a 96-well plate and incubated overnight. After incubation, the coating solution is removed, plates are washed, and wells are blocked with gelatin in PBS Tween-20® (PBS-TG). Both serum and whey are serially diluted in four-fold increments. Horseradish peroxidase (HRP)-conjugated polyclonal sheep anti-bovine IgG (1:10000) and polyclonal sheep anti-bovine IgG1 and IgG2 (1:5000) are added (100 μL) to each well and incubated for 1 h. After washing, 1×HRP substrate is added (100 μL) per well and incubated for 20 min at room temperature. Absorbance is read at a wavelength of 405 nm (A405) using a Synergy H1 Microplate Reader (Biotek Instrument Inc.). Data is exported to Excel (Microsoft Corporation, Redmond, WA) and the blank row's average+2 standard deviations (avg+2stdev) is used to calculate the cutoff point for titer calculation. Both serum and milk titers are calculated by the intersection of least-square regression of A405 versus the logarithm of the dilution. Cytokines are tested using bovine ELISA kits (Kingfisher Biotech, Inc., Saint Paul, MN) following manufacturer instructions.

### Example 15 Milk Leukocyte Isolation

About 125 ml of milk is collected from each mammary quarter and combined (500 mL). Milk leukocytes are isolated as described by others (Kimura et al., 2008, Mehrzad et al., 2008) with modifications. Briefly, milk is collected aseptically and placed immediately on ice and processed within 4 h. Samples are diluted 1:1 with PBS with 10% EDTA, centrifuged, and the top cream fat layer removed. The cells pellet is resuspended in PBS (pH7.4) with 10% EDTA and centrifuged as above. This procedure is repeated twice. The supernatant is removed by aspiration. Cell pellets are resuspended in PBS.

### Example 16 Total RNA Isolation and Quantitative Real Time PCR (qRT-PCR)

The total RNA isolation and analysis of cytokines by qRT-PCR is conducted as described elsewhere (Singh et al., 2008, Moyes et al., 2009, Kerro Dego et al., 2018) with modification. Briefly, total RNA is isolated from peripheral blood mononuclear cells (PBMC) and milk leukocytes using a total RNA isolation kit. The cDNA of IFNγ, IL-4, IL-10, IL-12, and IL17A, IL-17F, IL-22 is synthesized using gene specific primers by RT-PCR as described (Singh et al., 2008, Moyes et al., 2009). Expression of targeted cytokines relative to a housekeeping gene is analyzed by qRT-PCR using each gene-specific primers. Amplification is carried out in a total volume of 25 μl in 96 well plates using a Quant studio 7 (ABI, life technologies). The reaction is carried out using TaqMan probes and master mix (Applied Biosystems) following the manufacturer's instructions using 3 step amplification cycles: 950C for 15 sec, 550C for 30 sec, and 72° C. for 30 sec. The relative difference in gene expression is calculated as fold change using relative standard curve and the formula 2-ΔΔct (Livak and Schmittgen, 2001). The gene expression pattern of some of the known housekeeping genes such as β-actin, ubiquitin, GTP, UXT, MRPL39, DNASE1, NENF, and GRHL1 (Singh et al., 2008, Moyes et al., 2009) is analyzed and the most stable gene is selected for internal control in qRT-PCR reactions.

### Example 17 Challenge Infection

At 35 days in milk post calving, cows are challenged by Staph. aureus (Merrill et al., 2019) or Strep. uberis (Kerro Dego et al., 2021) as described previously. After challenge, rectal temperature, clinical assessment and scoring of inflammatory changes in the mammary glands and milk are conducted daily during the 7 days of challenge as described by (Merrill et al., 2019). Inflammatory changes in milk are scored as: 0=Normal, 1=Flakes, 2=Clots, 3=Stringy/watery/bloody. Inflammatory changes in the mammary gland tissue are scored as: 0=Normal; 1=Slight swelling; 2=Moderate swelling; the udder is firm, redness and heat detected, discomfort detected, 3=Severe swelling; the udder is very hard, red, and hot. Rectal temperatures are taken daily using a digital thermometer during the 7 days of challenge to monitor any potential systemic reaction. Clinically infected cows or quarters are treated with an antibiotic (Ceftiofur hydrochloride/Spectramast® LC) (Zoetis Inc., Kalamazoo, MI) following manufacturer instructions for lactating cow treatment. If the infection is not cured by treatment, the animal is removed from the study and culled.

### Example 18 Evaluation of Antibody Titers and Cytokines in Milk and Blood

Antibody titers in blood and milk after challenge infection are measured immediately before challenge at 35 DIM and weekly for 4 weeks at 42, 49, 56, and 63 DIM post challenge. Antibody titers in the milk and serum are determined by ELISA as described under 2.5. Proinflammatory Cytokine expression patterns in the blood and milk are tested during the first 3 consecutive days of challenge or post challenge by cytokine ELISA kit (KingFisher). Proinflammatory cytokines such as IL1B, IL-8, TNF-α, IL6, as well as cellular immunity-associated cytokines in the secretory glandular mucosa region such as IL17A, IL-17F, IL-22, are measured by cytokine ELISA kit.

### Example 19. Statistical Analysis

Quantitative outcomes of antibody responses and cytokine profiling are analyzed with mixed effects linear regression models by including treatment, time, and treatment by time interaction as fixed effects while cow ID is included as a random effect to account for the repeated measures within a cow. To better discern potential differences that exist in the response variables with respect to experimental infection status (clinical, subclinical, uninfected), additional analyses are conducted that include the fixed effect of status. A significant effect is declared when P≤0.05. Multiple comparisons among three infection statuses in the second model are compared using Fisher's least significant difference (LSD). Identifying response factors that lead to greater protection would be invaluable for helping the design of future vaccine formulations which promote that type of immune response. To accomplish this, multivariate analysis is conducted to identify combinations of measured responses (e.g. isotype titer, cytokine profiles, etc.) at all measured time points (e.g. 0, 28, 42 days after first vaccination). Analyses are conducted within a treatment group to evaluate if the combination of factors changes with vaccination against staphylococcal mastitis, so as to define the booster vaccination frequency. Between treatment groups analysis is used to identify the optimum vaccine dose. Statistical analysis are conducted SAS 9.4 and in STATA (StataCorp LLC, College Station, Texas).

### Example 20 Polyvalent Vaccine Preparation

Polyvalent vaccines that contain either 200 μg or 400 μg of each of the SASP, SCSP and SUSP vaccines are prepared. Efficacy of the triple S polyvalent vaccines is evaluated by controlled experimental challenge infection with either S. aureus, S. uberis, or a combination thereof.

### Example 21. Analysis of Individual Proteins in Surface-Associated Proteins

Immunogenic proteins in the SCSP and SASP vaccines were analyzed. Elongation factor (EF)-Tu (FAT), enolase (ENO), fructose-bisphosphate aldolase (FBA), phosphoglycerate kinase (FGQ), and glyceraldehyde-3-phosphate dehydrogenase are the among most immunogenic proteins in the SCSP and SASP vaccines. Of these proteins, interestingly, we found that three proteins, including EF-Tu, ENO, and FBA are also major immunogenic proteins in the Streptococcus uberis surface proteins (SUSP).

That which is claimed:

1. A pharmaceutical composition comprising isolated surface-associated polypeptides, wherein said surface associated polypeptides are selected from the group consisting of Staphylococcus aureus surface-associated polypeptides (SASP), Staphylococcus chromogenes surface-associated polypeptides (SCSP) and Streptococcus uberis surface associated polypeptides (SUSP).

2. The pharmaceutical composition of claim 1, comprising at least two isolated surface-associated polypeptides selected from the group consisting of Staphylococcus aureus surface-associated polypeptides (SASP), Staphylococcus chromogenes surface-associated polypeptides (SCSP) and Streptococcus uberis surface associated polypeptides (SUSP).

3. The pharmaceutical composition of claim 1, comprising isolated SASP, SCSP and SUSP.

4. The pharmaceutical composition of claim 1, wherein the composition further comprises an adjuvant capable of stimulating the immune response in a dairy producing mammal, selected from the group consisting of dairy cows, sheep, goats, camels, buffaloes, yaks, horses, reindeer and donkeys.

5. The pharmaceutical composition of claim 1, comprising at least about 0.05 mg of surface associated polypeptides selected from the group consisting of SASP and SCSP.

6. The pharmaceutical composition of claim 1, wherein the composition confers long lasting resistance to an intramammary infection (IMI).

7. The pharmaceutical composition of claim 1, wherein the composition inhibits bacterial infection in a subject to which the composition has been administered.

8. The pharmaceutical composition of claim 1, wherein the surface associated polypeptide is present in an effective amount to reduce the somatic cell count (SCC) in milk from a subject treated with the pharmaceutical composition.

9. The pharmaceutical composition of claim 1, wherein a subject to whom the pharmaceutical composition has been administered exhibits reduced risk of a disorder selected from the group consisting of intramammary infection, clinical mastitis, subclinical mastitis, and persistent intramammary infection.

10. A polyvalent vaccine against intramammary infection comprising at least two isolated surface-associated polypeptides selected from the group consisting of Staphylococcus aureus surface-associated polypeptides (SASP), Staphylococcus chromogenes surface-associated polypeptides (SCSP) and Streptococcus uberis surface associated polypeptides (SUSP).

11. The polyvalent vaccine of claim 10, wherein the isolated surface associated polypeptides are present at a predetermined ratio, wherein the predetermined ratio of surface-associated polypeptides is selected from the group consisting of a ratio of approximately 1:1:1 and a ratio not approximately 1:1:1.

12. The polyvalent vaccine of claim 10, comprising surface-associated polypeptides in an amount selected from the group consisting of (a) between about 50 μg and about 5000 μg surface-associated polypeptides; (b) between about 100 μg and about 3000 μg surface-associated polypeptides; and (c) between about 150 μg and about 2000 μg surface-associated polypeptides.

13. A method of inhibiting bacterial infection in a subject comprising administering a pharmaceutical composition comprising isolated surface-associated polypeptides to the subject, wherein said surface associated polypeptides are selected from the group consisting of Staphylococcus aureus surface-associated polypeptides (SASP), Staphylococcus chromogenes surface-associated polypeptides (SCSP) and Streptococcus uberis surface associated polypeptides (SUSP).

14. The method of claim 13, wherein the bacterial infection is an intramammary infection.

15. A method of reducing mastitis in a subject comprising administering a pharmaceutical composition of claim 1 to the subject.

16. The method of claim 15, wherein after administration of the pharmaceutical composition, the subject exhibits a decreased risk selected from the group comprising a decreased risk of clinical mastitis, a decreased risk of subclinical mastitis, a decreased risk of subclinical mastitis at the quarter level, a decreased risk of subclinical mastitis at the cow level.

17. A method of increasing antibody titers in milk comprising administering a pharmaceutical composition of claim 1 to a milk producing subject.

18. A method of reducing somatic cell count (SCC) in milk, comprising administering a pharmaceutical composition of claim 1 to a milk producing subject.

19. A method of reducing the incidence of subclinical mastitis in a herd of dairy producing mammals comprising the steps of: (a) providing a herd of dairy producing mammals and (b) administering a pharmaceutical composition of claim 1 to members of the herd.

20. A method of increasing milk yield in a dairy mammal comprising (a) providing a dairy mammal and (b) administering a pharmaceutical composition of claim 1 to the dairy mammal.
